(19) United States
(12) Patent Application Publication
(10) Pub. No.: US 2026/0056199 A1
(43) Pub. Date: Feb. 26, 2026

(54) METHOD AND KIT FOR DETECTING STAGE OF COLORECTAL HEALTH STATUS

(71) Applicant: Hangzhou Guangkeande Biotechnology Co., Ltd., Hangzhou (CN)

(72) Inventors: Junli GAO, Hangzhou (CN); Junshun Gao, Hangzhou (CN); Mingjie Tong, Hangzhou (CN); Xiaojun Peng, Hangzhou (CN); Qinqin Lou, Hangzhou (CN)

(21) Appl. No.: 18/918,510

(22) Filed: Oct. 17, 2024

(30) Foreign Application Priority Data
Aug. 20, 2024 (CN) 2024111470697
Aug. 20, 2024 (CN) 2024111530965

(51) Int. Cl. G01N 33/574; G01N 33/68

(52) U.S. Cl. CPC G01N 33/57419; G01N 33/6854; G01N 2800/56

(57) ABSTRACT

Disclosed is a method for diagnosing or predicting a stage of colorectal health status in an individual, including: providing a biological fluid sample of the individual; detecting the content of a marker in the biological fluid sample; and determining the stage of the colorectal health status in the individual according to the detected content of the marker, where the biomarker is selected from: any one or more of trefoil factor 1, trefoil factor 3, insulin-like growth factor binding protein 1, insulin-like growth factor binding protein 4, serine protease inhibitor A1, osteopontin, or growth differentiation factor-15.

### CROSS-REFERENCE TO RELATED APPLICATIONS

This application claims priority to Chinese Patent Applications, Application No.: 202411147069.7, filed on Aug. 20, 2024, Application No.: 202411153096.5, filed on Aug. 20, 2024; and all disclosures of this application, including but not limited to the abstract, claims, accompanying drawings, and specification of this application are incorporated by reference in their entirety as a part of this application.

### BACKGROUND OF THE INVENTION

### Field of the Invention

The present invention relates to the field of early diagnosis of colorectal cancer, and specifically to a biomarker for detecting early carcinogenesis of colorectum and an application thereof.

### Description of the Related Art

Colorectal cancer is one of the most common malignant tumors in clinical practice. According to the statistics of the National Cancer Center, the morbidity and mortality of colorectal cancer are among the top 5 malignant tumors. The morbidity of colorectal cancer is also increasing year by year due to various factors such as population aging and changes in the dietary structure. At present, the morbidity rate of colorectal cancer in China is surging at a rate of 4% every year, far exceeding the global average annual growth rate; on the contrary, the 5-year survival rate of colorectal cancer patients in China is only 47%, far lower than that in developed countries such as Europe and America.

Detecting and identifying malignant colorectal lesions in the early stage is an important measure to reduce the morbidity and mortality of colorectal cancer in China and improve the survival of colorectal cancer. Patients found and diagnosed with adenoma and stage I colorectal cancer after radical surgery have a 5-year survival rate of over 90% after radical surgery. However, the 5-year survival rate for patients who have already experienced distant metastasis at the time of discovery is only about 5%. Currently, colonoscopy is the gold standard for detecting colorectal tumors. However, colonoscopy requires advanced instruments and equipment and specialized operators, with high technical requirements and high costs. Moreover, subjects need intestinal preparation and have poor compliance, so colonoscopy is not suitable for repeated examination and population census. In addition, there are other methods for screening bowel cancer, such as the fecal occult blood test (FOBT), but this method has limitations such as susceptibility to dietary interference, a high false positive rate and low sensitivity. At present, the commonly used blood markers of colorectal cancer (CEA, CA199) are not ideal in the diagnosis of early-stage colorectal cancer and precancerous lesions (advanced adenomas), with insufficient sensitivity and a high false positive rate. Although some molecular diagnostic techniques based on blood and feces (such as DNA methylation detection) have improved the detection sensitivity of early-stage colorectal cancer to a certain extent, the diagnostic sensitivity for advanced adenomas is only about 60%. There is a lack of clinical biomarkers for early diagnosis of colorectal cancer, and especially the discovery of highly sensitive biomarkers for the diagnosis of advanced adenomas is of great significance.

Proteomics is a science that studies the composition, localization, changes, and interaction of proteins in cells, tissues, or organisms, including the study of protein expression patterns and proteomic functional patterns. With the development of proteomics technology, high performance liquid chromatography-high resolution tandem mass spectrometry has gradually become the mainstream technology of proteomics, and more and more novel tumor markers have been discovered. Although there have been many articles and patent reports on the discovery of novel tumor markers in recent years, they have only remained in the laboratory research stage and have little clinical application and market promotion. Moreover, in most cases, a single indicator is far from enough for in vitro diagnosis of tumors. Only by combining various dimensions of detection in the form of combined joint inspection can the accuracy of prediction be enhanced. Therefore, it is of great clinical value to search for novel markers related to early diagnosis of colorectal cancer and its precancerous lesions, and to combine multiple markers to construct early prediction models.

Therefore, the present invention screens protein markers related to diagnosis of early carcinogenesis of colorectum through high-throughput proteomics technology, and combines multiple markers to construct a prediction model of early carcinogenesis of colorectum, which will be of great significance for the early diagnosis and treatment of colorectal cancer.

### BRIEF SUMMARY OF THE INVENTION

In response to the problems existing in the prior art, the present invention provides a biomarker for detecting early carcinogenesis of colorectum and an application thereof. Utilizing the method of proteomics, by analyzing proteins with significant differences in abundance levels in the blood of patients with advanced adenomas, patients with early-stage colorectal cancer and healthy control populations in the progression of colorectal cancer, biomarkers that can be used to predict whether individuals suffer from early carcinogenesis of colorectum (including advanced adenomas and early-stage colorectal cancer) and for risk prediction are screened, and a multi-marker joint detection model is further constructed, which can accurately, non-invasively and efficiently predict the risk of early carcinogenesis of colorectum in individuals and meet clinical needs.

In one aspect, the present invention provides a method for predicting whether an individual suffers from early carcinogenesis of colorectum, including providing a biological fluid sample of the individual, testing a concentration or a content of a marker in the biological fluid sample to obtain the content of the marker, and determining whether the individual is in the early carcinogenesis of colorectum according to the content, where the marker is selected from: any one or more of trefoil factor 1, trefoil factor 3, insulin-like growth factor binding protein 1, insulin-like growth factor binding protein 4, serine protease inhibitor A1, osteopontin, and growth differentiation factor-15.

In some embodiments, the early carcinogenesis of colorectum includes an advanced adenoma and/or early-stage colorectal cancer.

In the present invention, the early carcinogenesis of colorectum is defined as including colorectal cancer and/or an advanced adenoma, that is, the marker provided by the present invention can simultaneously distinguish a population suffering from an advanced adenoma and/or early-stage colorectal cancer from a healthy population. That is, the marker of the present invention can be used to distinguish the population with the advanced adenoma from the healthy population, and can also be used to distinguish the population with early-stage colorectal cancer from the healthy population.

Although existing markers for distinguishing early-stage colorectal cancer have also been reported, they can usually only be used to distinguish healthy populations from populations with early-stage colorectal cancer, and cannot distinguish patients with advanced adenomas in an earlier stage of pathological changes, resulting in the problem of insufficient detection sensitivity.

The marker provided by the present invention can simultaneously diagnose populations suffering from advanced adenomas and/or early-stage colorectal cancer, distinguish them from healthy populations, and thus can identify malignant colorectal lesions in an earlier stage, which is of great significance for improving the survival rate of colorectal cancer.

Furthermore, the marker includes a combination mode of trefoil factor 1, trefoil factor 3, insulin-like growth factor binding protein 1, insulin-like growth factor binding protein 4, serine protease inhibitor A1, osteopontin, and growth differentiation factor-15.

The present invention, by utilizing the method of proteomics, collects plasma samples of patients in different stages of the tumor progression of colorectal cancer (the progression including inflammatory diseases, benign polyps, advanced adenomas, and early-stage colorectal cancer) and a healthy control population, analyzes different samples through high performance liquid chromatography-tandem mass spectrometry technology (HPLC-MS/MS), first screens proteins with significant differences between patients with early-stage colorectal cancer and the healthy control population based on orthogonal partial least squares discriminant analysis and significance analysis methods, and finally obtains 12 differential proteins with significant associations with colorectal cancer through screening. However, when these 12 proteins are adopted to construct a random forest model to distinguish the population with early carcinogenesis of colorectum (including early-stage colorectal cancer (CRC) and advanced adenomas (AA)) from the healthy population, the diagnostic efficiency is low, and in particular, it cannot be used to distinguish the population with advanced adenomas from the healthy population.

Therefore, in order to improve the diagnostic efficiency of early carcinogenesis of colorectum (including early-stage colorectal cancer and advanced adenomas) or advanced adenomas, differential proteins are screened for the advanced adenoma and healthy groups again, the top 10 differential proteins by importance ranking are finally obtained through screening, and 7 protein markers are finally obtained through screening by further using the Boruta algorithm to construct a model, which has a good risk prediction ability in the groups of early-stage colorectal cancer, advanced adenomas and early carcinogenesis of colorectum (including early-stage colorectal cancer and advanced adenomas), and can effectively distinguish these patients from healthy people.

In some embodiments, the body fluid sample includes any one or more of saliva, sweat, blood, urine, and spinal fluid.

In some embodiments, some reagents are used to test or detect the concentration or the content of the marker present in the sample.

In some ways, the reagent for prediction of suffering from early carcinogenesis of colorectum is a detection reagent prepared with the biomarker as the detection target. These detection reagents may include biological reagents and kits suitable for detecting the biomarker, such as sample pretreatment reagents, antigens or antibodies; and the detection reagents may also be developed into standardized reagents or kits suitable for liquid chromatography-ultraviolet (LC-UV) or liquid chromatography-mass spectrometry (LC-MS) detection of the biomarker.

In some embodiments, an ELISA method is adopted to perform cohort validation on a total of 18 candidate protein markers for colorectal cancer, including 12 candidate protein markers screened in early-stage colorectal cancer and 10 candidate protein markers screened in advanced adenomas (with 4 markers overlapping), and it is finally found that in two groups with early-stage colorectal cancer vs. healthy controls and advanced adenomas vs. healthy controls, 7 novel biomarkers (TFF1, TFF3, IGFBP1, IGFBP4, SERPINA1, OPN, GDF-15) with significant differences are found, which can serve as candidate biomarkers for differential diagnosis of early carcinogenesis of colorectum (including early-stage colorectal cancer and advanced adenomas) and advanced adenomas; at the same time, the model constructed with 7 protein markers has a good risk prediction ability in the early-stage colorectal cancer, advanced adenoma and early-stage colorectal cancer+advanced adenoma (early carcinogenesis of colorectum) groups, where in the early carcinogenesis of colorectum vs. healthy control groups, the AUC value of the model reaches 0.896; in the early-stage colorectal cancer vs. healthy control (HC) groups, the AUC value of the model reaches 0.983; and in the advanced adenoma vs. healthy control groups, the AUC value of the model reaches 0.807, and the AUC values all reach above 0.8, indicating high diagnostic value; and the 7 markers that contribute significantly to the model finally obtained through screening are all differential markers contained in advanced adenomas, and the optimal 7 markers that contribute significantly to the model in the present invention cannot be obtained by screening only from the protein markers of early-satge colorectal cancer.

Therefore, the preferred 7 markers in the present invention are: trefoil factor 1 (TFF1), trefoil factor 3 (TFF3), insulin-like growth factor binding protein 1 (IGFBP1), insulin-like growth factor binding protein 4 (IGFBP4), serine protease inhibitor A1 (SERPINA1), osteopontin (OPN), and growth differentiation factor-15 (GDF-15). The diagnostic model constructed using these 7 markers has good clinical diagnostic value for early carcinogenesis of colorectum, and can significantly improve the diagnostic differential ability and diagnostic efficiency of early carcinogenesis of colorectum (including early-stage colorectal cancer+advanced adenomas) or advanced adenomas, and realizes the risk prediction of early carcinogenesis of colorectum (including early-stage colorectal cancer+advanced adenomas) or advanced adenomas.

Furthermore, the reagent is used to detect the presence or absence, or relative abundance or concentration of the biomarker in the body fluid sample.

The present invention screens biomarkers for early carcinogenesis of colorectum or advanced adenomas from blood, and these biomarkers have significant differences in blood of the population with early carcinogenesis of colorectum (including early-stage colorectal cancer+advanced adenomas) and non-colorectal cancer population (including but not limited to benign polyps, patients with gastrointestinal inflammatory diseases, healthy controls). By collecting blood samples, these biomarkers in the blood of an individual can be detected to predict or assist in diagnosing the possibility of early carcinogenesis of colorectum or advanced adenomas in the individual, or these biomarkers in the blood of a certain group can be detected, and then the group can be divided into a high-risk population with colorectal canceration or a low-risk population with colorectal canceration.

Furthermore, a method for the detection includes a radiometric method, an immune method, a fluorescence method, a flow fluorescence method, a latex turbidimetry method, a biochemical method, an enzymatic method, a hybridization method, a gas chromatography-mass spectrometry method, a liquid chromatography-mass spectrometry method, a chromatography method, a chemiluminescence method, a magnetoelectric method, or a photoelectric conversion method, etc. to test the content or the concentration of the marker.

The presence or absence of the marker here or the level of the marker content is a relative concept. For example, for the comparison between the diseased group and the non-diseased group, the content of these specific markers is compared with the diseased or non-diseased group as a benchmark. It may be certain markers that have higher levels in the diseased group compared with the non-diseased group, and this higher level has a statistical difference, e.g. with a significant or an extremely significant increase. Therefore, when these markers are used for determination, if a single marker is used, if the content of the marker changes when the probability of a certain risk increases, the change here may be a relative increase or a relative decrease. The difference of this relative increase or decrease is significant, or even extremely significant. Therefore, in some embodiments, the content or the concentration of the marker obtained in the test sample is compared with a preset threshold value, and the result of the comparison is used to determine the status of the individual, particularly the health status of the colorectum. No matter what method is used for detection, a predetermined value (cut-off value) can be used as the standard. If a value is higher than this value, it is considered that the content changes. Having such a result can have predictive or diagnostic value.

Therefore, in some aspects, in the method of the present invention, the content of the marker in the sample can be detected by any existing known method, such as liquid chromatography, gas chromatography, mass spectrometry, LC-MS, gas chromatography-mass spectrometry (GC-MS), chromatography-mass spectrometry (CC-MS), liquid chromatography-tandem mass spectrometry (LC-MS-MS), nuclear magnetic resonance spectroscopy (NMR), immunochromatographic test strips, immune reaction chips, capillary electrophoresis, infrared spectroscopy, etc., and as long as it can be used to detect the content of the protein marker in the sample, it can be used for the diagnosis of early carcinogenesis of colorectum and advanced adenomas. As long as the content of the protein marker in the sample can be detected, it can be used to predict or diagnose the probability of the occurrence of a certain disease. It can be understood that the detection here is to detect an individual sample, and then compare it with a preset standard (Cut-off). The result of the comparison is used to determine or predict the occurrence status of the disease. For example, it can be used to predict the probability of the occurrence of early carcinogenesis of colorectum or advanced adenomas. This prediction or diagnosis is based on whether the disease occurs within a certain time. Of course, this detection can be continuous detection, and the progression of disease occurrence can be inferred with changes in the contents of certain substances.

In some embodiments, the relative abundance is the peak area of the biomarker in a detection spectrum obtained by high performance liquid chromatography-tandem mass spectrometry. For example, if the average peak area of a certain biomarker measured in the control sample is 500, and the average peak area measured in the sample of patients with early carcinogenesis of colorectum or advanced adenomas is 3000 or higher than 3000, then it is considered that the abundance of the biomarker in the sample is 6 times that of the control sample.

In some embodiments, the marker includes a combination form of trefoil factor 1, trefoil factor 3, insulin-like growth factor binding protein 1, insulin-like growth factor binding protein 4, serine protease inhibitor A1, osteopontin, and growth differentiation factor-15. In some embodiments, this combination may also be combined with other markers or other parameters for diagnosis.

In a second aspect, the present invention provides a kit for predicting whether an individual suffers from early carcinogenesis of colorectum, including a detection reagent for detecting a biomarker according to any one of the above technical solutions.

In some embodiments, the detection reagent is an antibody to the biomarker, and the antibody is a monoclonal antibody.

In yet another aspect, the present invention provides a system for predicting whether an individual suffers from early carcinogenesis of colorectum, including a data analysis module, where the data analysis module is configured to analyze the detection value of a biomarker, the marker includes any one or more of trefoil factor 1, trefoil factor 3, insulin-like growth factor binding protein 1, insulin-like growth factor binding protein 4, serine protease inhibitor A1, osteopontin, and growth differentiation factor-15, and the early carcinogenesis of colorectum includes advanced adenomas and early-stage colorectal cancer.

Furthermore, the marker includes trefoil factor 1, trefoil factor 3, insulin-like growth factor binding protein 1, insulin-like growth factor binding protein 4, serine protease inhibitor A1, osteopontin, and growth differentiation factor-15.

In some embodiments, random forest is first adopted to construct a model, and it is preliminarily confirmed that any one of the 7 novel biomarkers screened is adopted alone, and the concentration changes of any one of the biomarkers can be used to distinguish between patients with early carcinogenesis of colorectum (early-stage colorectal cancer+advanced adenomas) and the healthy population, patients with advanced adenomas and the healthy population, and patients with early-stage colorectal cancer and the healthy population, indicating that these 7 biomarkers have extremely high diagnostic value.

To sum up, it can be seen that the joint diagnostic model including the 7 biomarkers (trefoil factor 1 (TFF1), trefoil factor 3 (TFF3), insulin-like growth factor binding protein 1 (IGFBP1), insulin-like growth factor binding protein 4 (IGFBP4), serine protease inhibitor A1 (SERPINA1), osteopontin (OPN), and growth differentiation factor-15 (GDF-15)) constructed in this example has good clinical diagnostic value for early carcinogenesis of colorectum, can significantly improve the diagnostic differentiation ability and diagnostic efficiency for early carcinogenesis of colorectum, early-stage colorectal cancer or advanced adenomas, and achieves accurate diagnosis for early carcinogenesis of colorectum, early-stage colorectal cancer or advanced adenomas.

Furthermore, the data analysis module adopts the detection values of the markers of the known samples as a training set, divides them into an early carcinogenesis of colorectum group and a healthy group according to whether the individuals suffer from early carcinogenesis of colorectum, analyzes the relationship between the detection values of the early carcinogenesis of colorectum group and the healthy group, and constructs a model.

Furthermore, the system further includes a data storage module, a data input interface and a data output interface; the data storage module is configured to store the detection value of the biomarker; and the data input interface is configured to input the detection value of the biomarker, and the data output interface is configured to output a prediction result.

Furthermore, the detection value is the presence or absence or relative abundance or concentration value of the 7 biomarkers.

In yet another aspect, the present invention provides an application of a biomarker for preparation of a reagent for predicting whether an individual suffers from an advanced adenoma, where the biomarker includes any one or more of trefoil factor 1, trefoil factor 3, insulin-like growth factor binding protein 1, insulin-like growth factor binding protein 4, serine protease inhibitor A1, osteopontin, growth differentiation factor-15, prion protein, guanylate cyclase activator 2A, and regenerating family member protein 1a.

Furthermore, the biomarker includes trefoil factor 1, trefoil factor 3, insulin-like growth factor binding protein 1, insulin-like growth factor binding protein 4, serine protease inhibitor A1, osteopontin, and growth differentiation factor-15.

Furthermore, the reagent is used to detect the content of the biomarker in the body fluid sample; and the body fluid sample includes any one or more of saliva, blood, urine, plasma, serum, and spinal fluid.

Furthermore, the reagent is used to detect the presence or absence, or relative abundance or concentration of the biomarker in the body fluid sample.

Furthermore, a method for the detection includes a radiometric method, an immune method, a fluorescence method, a flow fluorescence method, a latex turbidimetry method, a biochemical method, an enzymatic method, a hybridization method, a gas chromatography-mass spectrometry method, a liquid chromatography-mass spectrometry method, a chromatography method, a chemiluminescence method, a magnetoelectric method, or a photoelectric conversion method.

In yet another aspect, the present invention provides a combination of biomarkers for predicting whether an individual suffers from an advanced adenoma, the combination including trefoil factor 1, trefoil factor 3, insulin-like growth factor binding protein 1, insulin-like growth factor binding protein 4, serine protease inhibitor A1, osteopontin, and growth differentiation factor-15.

In a third aspect of the present invention, the present invention provides a kit for predicting whether an individual suffers from an advanced adenoma, including a detection reagent for a biomarker for use according to any one of the above technical solutions.

In yet another aspect, the present invention provides a system for predicting whether an individual suffers from an advanced adenoma, including a data analysis module, where the data analysis module is configured to analyze the detection value of a biomarker, and the biomarker includes any one or more of trefoil factor 1, trefoil factor 3, insulin-like growth factor binding protein 1, insulin-like growth factor binding protein 4, serine protease inhibitor A1, osteopontin, growth differentiation factor-15, prion protein, guanylate cyclase activator 2A, and regenerating family member protein 1a.

Furthermore, the biomarker includes trefoil factor 1, trefoil factor 3, insulin-like growth factor binding protein 1, insulin-like growth factor binding protein 4, serine protease inhibitor A1, osteopontin, and growth differentiation factor-15.

Furthermore, the data analysis module adopts the detection values of the markers of the known samples as a training set, divides them into an advanced adenoma group and a healthy group according to whether the individuals suffer from advanced adenomas, analyzes the relationship between the detection values of the advanced adenoma group and the healthy group, and constructs a model.

Furthermore, the system further includes a data storage module, a data input interface and a data output interface; the data storage module is configured to store the detection value of the biomarker; and the data input interface is configured to input the detection value of the biomarker, and the data output interface is configured to output a prediction result.

Furthermore, the detection value is the presence or absence or relative abundance or concentration value of the 7 biomarkers.

Furthermore, the trefoil factor 1 is a protein or an amino acid sequence with a UniProt database number of P04155; the trefoil factor 3 is a protein or an amino acid sequence with a UniProt database number of Q07654; the insulin-like growth factor binding protein 1 is a protein or an amino acid sequence with a UniProt database number of P08833; the insulin-like growth factor binding protein 4 is a protein or an amino acid sequence with a UniProt database number of P22692; the serine protease inhibitor A1 is a protein or an amino acid sequence with a UniProt database number of P01009; the osteopontin is a protein or an amino acid sequence with a UniProt database number of P10451; and the growth differentiation factor-15 is a protein or an amino acid sequence with a UniProt database number of Q99988.

In a fourth aspect of the present invention, the present invention provides a method for diagnosing whether an individual belongs to a population with an early-stage colorectal cancer, an advanced adenoma, a benign polyp, and an inflammatory bowel disease, a healthy population, or a population with other cancers, or a method for diagnosing colorectal health status of an individual, the method including providing a biological fluid sample of the individual, testing a content or a concentration of a marker in the biological fluid sample, and determining whether the individual belongs to a population with an early-stage colorectal cancer, an advanced adenoma, a benign polyp, and an inflammatory bowel disease, a healthy population, or a population with other cancers according to the concentration, where the biomarker is selected from: one or a combination of more than one of trefoil factor 1, leaf factor 3, insulin-like growth factor binding protein 1, insulin-like growth factor binding protein 4, serine protease inhibitor A1, osteopontin, and growth differentiation factor-15.

In some ways, the method of the present invention includes using the model constructed with the above markers for diagnosis, where the model is a binary classification model, for example, for distinguishing two classifications of “the healthy population and patients with early carcinogenesis of colorectum”, “the healthy population and patients with advanced adenomas”, or “the healthy population and patients with early-stage colorectal cancer”, etc.

The present invention also attempts to construct a senary classification model using the 7 protein markers, and the senary classifications include senary classifications of early colorectal cancer, advanced adenomas, benign polyps, inflammatory bowel diseases, healthy status, and other cancers, and it is found that the model constructed by the present invention also has excellent diagnostic efficiency in distinguishing the senary classifications. At the same time, a variety of different algorithms are compared to construct a senary classification model, and it is found that the senary classification detection model constructed by adopting the gradient boosting algorithm has the highest diagnostic efficiency, which can be used for efficient differential diagnosis of early-stage colorectal cancer (CRC), advanced adenomas (AA), benign polyps (BPs), inflammatory bowel diseases (IBDs), healthy status, and other cancers, that is, it can effectively distinguish between six different healthy populations.

The other cancers include other digestive tract cancers, such as esophageal cancer, gastric cancer, liver cancer, pancreatic cancer, bile duct cancer, etc. The senary classification detection model of the present invention can not only accurately distinguish early carcinogenesis of colorectum from early-stage colorectal cancer, advanced adenomas, benign polyps, inflammatory bowel diseases, and healthy status, but also show significant advantages in distinguishing early carcinogenesis of colorectum from other digestive tract cancers (such as esophageal cancer, gastric cancer, liver cancer, pancreatic cancer, bile duct cancer, etc.). Through this model, early carcinogenesis of colorectum can be clearly distinguished from other cancers, which fully reflects the high specificity of the model. This means that the model can accurately identify the unique characteristics of early carcinogenesis of colorectum and avoid confusing it with other similar conditions or other cancer types, thus greatly reducing the possibility of misdiagnosis.

At the same time, in order to further improve the performance and accuracy of the senary classification model, the preliminary comparison and screening are performed on the model supervised classification algorithm models. The final results show that the optimal model constructed by the gradient boosting machine (GBM) is selected as the final prediction model for the diagnosis of advanced adenomas or early carcinogenesis of colorectum. The performance evaluation score of this algorithm is the best, with comprehensive diagnostic accuracy of 0.768 and consistency of 0.713 for predicting each disease type. Moreover, through 10-fold cross-validation method training, the optimal hyperparameters of the model are determined: the learning rate is 0.1, the number of decision trees (number of trees) is 150, the maximum tree depth (max depth) is 3, and the minimum number of samples for the terminal node (min samples) is 10.

In order to maximize the benefits while ensuring the diagnostic efficiency of the model, different quantities of protein marker combinations are further screened. Finally, 7 markers are preferred: trefoil factor 1 (TFF1), trefoil factor 3 (TFF3), insulin-like growth factor binding protein 1 (IGFBP1), insulin-like growth factor binding protein 4 (IGFBP4), serine protease inhibitor A1 (SERPINA1), osteopontin (OPN), and growth differentiation factor-15 (GDF-15) to construct a diagnostic model. At this time, the model can be constructed with the fewest markers to achieve the highest diagnostic efficiency.

The multi-classification model based on the selected gradient boosting algorithm is used for more in-depth prediction analysis. The predicted probability value is calculated, and the diagnostic indexes of each disease are re-given, so as to more accurately determine the diagnostic performance and threshold value of the model in different disease classifications. The multi-classification model of the gradient boosting machine (GBM) algorithm in the model group is used for prediction analysis, and the prediction results are calculated as the predicted probability values of 6 classifications (healthy controls, inflammatory bowel diseases, benign polyps, advanced adenomas, early-stage colorectal cancer, and other cancers), where the classification with the largest predicted probability value is the final prediction result of the system. The final result is that the model has an accuracy of 0.761 and a consistency of 0.705 in the model group. For early-stage colorectal cancer, the diagnostic sensitivity is 76.9%, specificity is 95.9%, positive predictive value is 78.4%, and negative predictive value is 95.5%; for advanced adenomas, the diagnostic sensitivity is 69.8%, specificity is 94.5%, positive predictive value is 71.3%, and negative predictive value is 94.1%; for benign polyps, the diagnostic sensitivity is 74.3%, specificity is 95.4%, positive predictive value is 67.7%, and negative predictive value is 96.6%; for inflammatory bowel diseases, the diagnostic sensitivity is 67.2%, specificity is 94.2%, positive predictive value is 67.7%, and negative predictive value is 94.1%; for other cancers, the diagnostic sensitivity is 77.7%, specificity is 95.9%, positive predictive value is 67.3%, and negative predictive value is 97.5%; and for healthy controls, the diagnostic sensitivity is 83.6%, specificity is 95.4%, positive predictive value is 89.0%, and negative predictive value is 92.9%, which is significantly better than the effect of the combined prediction model of the existing biomarkers.

At the same time, in order to test the performance of the above gradient boosting algorithm in new sample validation set data, so as to more comprehensively evaluate the generalization ability and practical application effect of the model, the algorithm constructed based on the model group is applied to the new validation group to validate the predictive performance. In new validation group samples, the final results show that the accuracy is 0.78 and the consistency is 0.729. For early-stage colorectal cancer, the diagnostic sensitivity is 79.4%, specificity is 94.4%, positive predictive value is 73.5%, and negative predictive value is 95.9%; for advanced adenomas, the diagnostic sensitivity is 73.4%, specificity is 94.7%, positive predictive value is 73.4%, and negative predictive value is 94.7%; for benign polyps, the diagnostic sensitivity is 72.7%, specificity is 95.9%, positive predictive value is 69.6%, and negative predictive value is 96.5%; for inflammatory bowel diseases, the diagnostic sensitivity is 68.3%, specificity is 96.3%, positive predictive value is 77.4%, and negative predictive value is 94.3%; for other cancers, the diagnostic sensitivity is 83.3%, specificity is 96.0%, positive predictive value is 68.2%, and negative predictive value is 98.3%; and for healthy controls, the diagnostic sensitivity is 85.0%, specificity is 96.3%, positive predictive value is 91.1%, and negative predictive value is 93.5%.

In yet another aspect, the present invention provides a combination of biomarkers for predicting whether an individual suffers from early-stage colorectal cancer, an advanced adenoma, a benign polyp, or an inflammatory bowel disease, is in healthy status, or suffers from other cancers, the combination including trefoil factor 1, trefoil factor 3, insulin-like growth factor binding protein 1, insulin-like growth factor binding protein 4, serine protease inhibitor A1, osteopontin, and growth differentiation factor-15.

In yet another aspect, the present invention provides a kit for predicting whether an individual suffers from an early-stage colorectal cancer, an advanced adenoma, a benign polyp, or an inflammatory bowel disease, is in healthy status, or suffers from other cancers, including a detection reagent for a biomarker for use according to any one of the above technical solutions.

In yet another aspect, the present invention provides a system for predicting whether an individual suffers from an early-stage colorectal cancer, an advanced adenoma, a benign polyp, or an inflammatory bowel disease, is in healthy status, or suffers from other cancers, including a data analysis module, where the data analysis module is configured to analyze the detection value of a biomarker, and the marker includes any one or more of trefoil factor 1, trefoil factor 3, insulin-like growth factor binding protein 1, insulin-like growth factor binding protein 4, serine protease inhibitor A1, osteopontin, and growth differentiation factor-15.

Furthermore, the marker includes trefoil factor 1, trefoil factor 3, insulin-like growth factor binding protein 1, insulin-like growth factor binding protein 4, serine protease inhibitor A1, osteopontin, and growth differentiation factor-15.

Furthermore, the data analysis module adopts the detection values of the markers of the known samples as a training set, divides them into an early-stage colorectal cancer group, an advanced adenoma group, a benign polyp group, an inflammatory bowel disease group, a healthy group, and another cancer group according to different disease classifications, analyzes the relationship among the detection values of the early-stage colorectal cancer group, the advanced adenoma group, the benign polyp group, the inflammatory bowel disease group, the healthy group, and the other cancer group, and constructs a model.

Furthermore, the system further includes a data storage module, a data input interface and a data output interface; the data storage module is configured to store the detection value of the biomarker; and the data input interface is configured to input the detection value of the biomarker, and the data output interface is configured to output a prediction result.

The beneficial effects of the present invention are as follows.

1. The present invention screens 7 novel biomarkers that can predict the risk of early carcinogenesis of colorectum (early-stage colorectal cancer+advanced adenomas) and advanced adenomas: trefoil factor 1, trefoil factor 3, insulin-like growth factor binding protein 1, insulin-like growth factor binding protein 4, serine protease inhibitor A1, osteopontin, and growth differentiation factor-15, and develops a new combination of protein markers, which can effectively evaluate and diagnose patients with advanced colorectal adenomas and colorectal cancer, effectively distinguishes between patients with colorectal cancer and the healthy population, patients with early-stage colorectal cancer and the healthy population, and patients with advanced adenomas and the healthy population, and can more accurately identify patients with early carcinogenesis of colorectum from patients with advanced adenomas. Compared with traditional detection methods, the present invention reduces the risk of misdiagnosis and missed diagnosis, and provides strong support for early detection and intervention of diseases.

2. In the present invention, the binary classification algorithm is adopted, and the model is constructed by the random forest algorithm, which shows significant diagnostic value in the comparison between early carcinogenesis of colorectum and healthy controls, early-stage colorectal cancer and healthy controls, and advanced adenomas and healthy controls; in the early carcinogenesis of colorectum vs. healthy control groups, the AUC value of the model reaches 0.896; in the early-stage colorectal cancer vs. healthy control groups, the AUC value of the model reaches 0.983; and in the advanced adenoma vs. healthy control groups, the AUC value of the model reaches 0.807, all of which exceed 0.8. Compared with the performance of the model constructed by the combination of traditional biomarkers for early-stage colorectal cancer (ROC in the advanced adenoma vs. healthy control groups is 0.699), the performance of the model herein increased by 10%, indicating that this model has higher diagnostic efficiency, which makes disease screening more efficient and accurate, can detect potential patients in time, and provides valuable opportunities for early treatment.

3. In the present invention, a senary classification algorithm is simultaneously adopted, a combined differential diagnostic model of the 7 biomarkers is constructed by the gradient boosting algorithm, and it is found that the diagnostic efficiency of the colorectal cancer diagnostic model constructed by adopting the 7 biomarkers including trefoil factor 1, trefoil factor 3, insulin-like growth factor binding protein 1, insulin-like growth factor binding protein 4, serine protease inhibitor A1, osteopontin, and growth differentiation factor-15 is optimal, and can be used to more efficiently predict early carcinogenesis of colorectum, and the model has accuracy of 0.78 and consistency of 0.729. For early-stage colorectal cancer, the diagnostic sensitivity is 79.4%, specificity is 94.4%, positive predictive value is 73.5%, and negative predictive value is 95.9%; for advanced adenomas, the diagnostic sensitivity is 73.4%, specificity is 94.7%, positive predictive value is 73.4%, and negative predictive value is 94.7%, which is significantly better than the effects of existing diagnostic models. This model significantly improves the accuracy, sensitivity and specificity of diagnosis, and can be used for efficient differential diagnosis of early-stage colorectal cancer, advanced adenomas, benign polyps, inflammatory diseases, healthy status, and other cancers, so as to provide timely intervention for patients; and the multi-classification model can provide clinicians with more comprehensive and accurate diagnosis basis, help to formulate personalized treatment plans, and improve the scientificity and effectiveness of medical decision-making.

4. The combined differential diagnostic model of the 7 biomarkers constructed in the present invention is convenient and fast, the detection result is highly consistent with the detection result of the clinical gold standard, and at the same time, this model significantly reduces the cost of diagnosing early carcinogenesis of colorectum and has a good application prospect.

5. The combined differential diagnostic model of the 7 biomarkers constructed in the present invention can used for accurate differential diagnosis of patients with early carcinogenesis of colorectum, so as to facilitate early detection and early intervention, promote early detection and early treatment of early carcinogenesis of colorectum, and meet urgent clinical needs.

### DETAILED DESCRIPTION

### (1) Diagnosis or Detection

Diagnosis or detection herein refers to detecting or assaying a biomarker in a sample, or content of a target biomarker, such as an absolute content or a relative content, and then indicating whether the individual providing the sample is likely to have or suffer from a certain disease, or the possibility of having a certain disease, by the presence or absence, or quantity of the target marker. The meanings of diagnosis and detection here are interchangeable. The result of this test or diagnosis cannot be directly used as a direct result of suffering from a disease, but is an intermediate result. If a direct result is obtained, other auxiliary methods such as pathology or anatomy are also needed to confirm that the individual suffers from a certain disease. For example, the present invention provides multiple novel biomarkers associated with early carcinogenesis of colorectum, and the changes in the contents of these biomarkers are directly associated with the presence or absence of colorectal cancer.

(2) Association of Markers or Biomarkers or Differential Proteins with Early Carcinogenesis of Colorectum or Advanced Adenomas

Markers, biomarkers and differential proteins have the same meaning in the present invention. The association here refers to the direct association between the appearance or content change of a certain biomarker in a sample and a specific disease, e.g. a relative increase or decrease in the content, indicating a higher possibility of suffering from this disease compared with a healthy population.

If multiple different markers appear simultaneously or the content changes relatively in the sample, it also indicates a higher possibility of suffering from this disease compared with a healthy population. That is to say, among the types of markers, certain markers have a strong association with suffering from a disease, some markers have a weak association with suffering from a disease, or some even have no association with a specific disease. One or more of those markers with a strong association can be used as markers for diagnosing a disease, and those markers with a weak association can be combined with the markers with a strong association to diagnose a certain disease, thereby increasing the accuracy of detection results.

For the numerous biomarkers found in the serum in the present invention, these markers can be used to distinguish colorectal cancer patients from healthy or benign disease populations. The markers here can be used as individual markers for direct detection or diagnosis, and selecting such markers indicates a strong association between the relative changes in the contents of the markers and early carcinogenesis of colorectum. Of course, it can be understood that one or more markers strongly associated with early carcinogenesis of colorectum can be selected for simultaneous detection. It is normally understood that in some embodiments, selecting strongly associated biomarkers for detection or diagnosis can achieve a certain standard of accuracy, such as 60%, 65%, 70%, 80%, 85%, 90%, or 95% accuracy, which can indicate that these markers can obtain an intermediate value for diagnosing a certain disease, but does not mean that suffering from a certain disease can be directly confirmed.

Of course, differential proteins with higher ROC values can also be selected as diagnostic markers. The so-called strong or weak association is generally calculated and confirmed by some algorithms such as the contribution rate or weight analysis of markers and colorectal cancer. Such calculation methods can be significance analysis (p value or false discovery rate (FDR) value) and fold change. Multivariate statistical analysis mainly includes principal component analysis (PCA), partial least squares discriminant analysis (PLS-DA) and orthogonal partial least squares discriminant analysis (OPLS-DA), and of course also includes other methods, such as ROC analysis. Of course, other model prediction methods are also possible. When specifically selecting biomarkers, the differential proteins disclosed in the present invention can be selected, or other existing known marker combinations can be selected or combined for prediction through model methods.

### (3) Definition of Disease Stages

Colorectal cancer stage: colorectal cancer, also known as large intestine cancer, refers to cancer originating from the epithelium of the large intestine, including colon cancer and rectal cancer. The most common pathological type is adenocarcinomas, and very few cases are squamous cell carcinoma. In China, rectal cancer is the most common, followed by colon cancer (sigmoid colon, cecum, ascending colon, descending colon, and transverse colon). The treatment of colorectal cancer should adopt the principle of individualized treatment. According to the patient's age, constitution, pathological type of tumor, and scope of invasion (stage), appropriate treatment methods should be selected, including radical surgery, chemotherapy, targeted therapy, radiotherapy, etc.; the formation of colorectal cancer generally goes through the development process of normal mucosal hyperplasia, advanced adenomas (malignant) and adenocarcinomas (malignant), which usually takes 5-10 years. Therefore, early screening, diagnosis and treatment are the most effective means to reduce the mortality of colorectal cancer. In particular, if intervention can be carried out at the stage of polyp adenomas (malignant), the occurrence of colorectal cancer can be effectively prevented. Advanced adenomas (AAs) are a kind of precancerous lesions that refer to a villous component or high degree of dysplasia of ≥1 cm in size or ≥25% of any size, which can easily develop into colorectal cancer over time. If tumor biomarkers with certain early warning effects can be found in the early stage of colorectal cancer occurrence to diagnose colorectal cancer and advanced adenomas (malignant tumors), it is of great significance to improve the treatment effect and prognosis of patients.

Early carcinogenesis of colorectum stage: in the present invention, early carcinogenesis of colorectum includes early-stage colorectal cancer and advanced adenomas.

Early-stage colorectal cancer stage: early-stage colorectal cancer refers to colorectal epithelial tumors of any size whose invasion depth is confined to the mucosa and submucosa, regardless of lymph node metastasis.

Colorectal adenoma stage: colorectal adenomas are benign tumors originating from the glandular epithelium of the colorectal mucosa, including colonic adenomas and rectal adenomas, and are common benign intestinal tumors. Adenomas can be classified into tubular adenomas, villous adenomas and tubulo-villous adenomas based on their structural characteristics. Methods such as endoscopic high-frequency electrocoagulation, laser and microwave coagulation can be adopted for resection, or surgical resection can be chosen, with regular follow-up. Those with malignant transformation choose other treatments (such as radiotherapy, chemotherapy, surgery, etc.) according to their condition.

Advanced colorectal adenoma stage: advanced colorectal adenomas refer to tubulo-villous adenomas, villous adenomas and/or adenomas with high-grade dysplasia with a diameter >1 cm. Due to the close relationship with the occurrence of colorectal cancer, advanced colorectal adenomas are considered a kind of precancerous lesions. Advanced adenomas should be removed in time to prevent evolution into colorectal cancer.

Colorectal polyp stage: colorectal polyps bulge on the surface of the colorectal rectum, which can be adenomas or hyperplasia and hypertrophy of the intestinal mucosa, and are collectively referred to as polyps before the pathological nature is determined. Benign polyps refer to adenomas or masses diagnosed as benign by pathological diagnosis. Whether polyps need surgical resection is mainly related to the size, shape and nature of the polyps. 1. If the diameter of the polyps is less than 2 cm, surgical resection is generally not required, and regular colonoscopy reexamination is enough; 2. if the diameter of the intestinal polyps is greater than 2 cm, surgical resection is recommended; and 3. if the intestinal polyps have a tendency towards malignancy, regardless of the diameter of the polyps, surgical resection is recommended.

Inflammatory bowel disease stage (IBDs): inflammatory bowel diseases are a kind of non-specific chronic inflammatory diseases of the intestinal tract, mainly including ulcerative colitis and Crohn's disease. The former mainly damages the colon and rectum, while the latter can damage any part of the gastrointestinal tract from the mouth to the anus, with the end of the small intestine and the colon being more common. The treatment mainly includes drug therapy, endoscopic therapy, and surgical therapy, with the treatment goal of promoting the healing of the intestinal mucosa, reducing clinical symptoms, preventing related complications, improving the quality of life of patients, and preventing disease recurrence.

Healthy individuals: the healthy individuals here are those without gastrointestinal diseases (no colorectal cancer, adenomas, advanced adenomas, polyps, or inflammatory bowel diseases), as well as those without other major diseases or malignant cancers.

The above stages are relative, and the development of colorectal diseases can develop from a healthy individual to an inflammatory stage, and can also gradually develop from a polyp stage to an advanced adenoma stage.

Therefore, the predictive model provided by the present invention can quickly distinguish patients with early carcinogenesis of colorectum, patients with advanced adenomas, patients with benign polyps, patients with inflammatory bowel diseases, and healthy individuals based on changes in the concentrations of biomarkers in body fluid samples.

(4) Gold standard for the diagnosis of early carcinogenesis of colorectum: patients with early-stage colorectal cancer often have no symptoms or signs in clinical practice, and rely on standardized colonoscopy examination by qualified physicians for diagnosis, and biopsy histopathology is the basis for diagnosis.

### Antibody or Antibody Fragment

The antibody used in the present invention can be any antibody that can specifically bind to a marker in a sample. The antibody that can be used as a binding agent can be any antibody known to those skilled in the art. The “antibody” can be an immunoglobulin molecule and an antigen-binding portion of an immunoglobulin molecule, i.e. a molecule containing an antigen-binding site that specifically binds to a marker substance. This term also includes derivatives of antibodies in which the binding ability is retained, and also includes any protein containing a binding domain that is homologous or largely homologous to the binding domain of an immunoglobulin. These proteins may originate from natural substances, or they may be partially or fully synthesized. An antibody may be monoclonal or polyclonal. An antibody may be a member of any immunoglobulin type, including any human immunoglobulin type: IgG, IgM, IgA, IgD, IgG, and IgE. An “antibody fragment” is a derivative of an antibody or a portion of an antibody that is less than the full length. The antibody fragment can retain at least one significant site of the binding ability of the full-length antibody. Examples of the antibody fragment include an antigen-binding fragment (Fab), an Fab′, an F(ab′)₂, a single-chain variable fragment (scFv), a variable fragment (Fv), a disulfide-stabilized Fv (dsFv) dimer, and an Fd fragment, but are not limited to the above. The antibody fragment can be generated by any means. For example, the antibody fragment can be generated by enzymatic or chemical cleavage of a complete antibody, or can also be generated by recombination of genes encoding partial antibody sequences. In other words, the antibody fragment can be generated by partial or complete recombination. The antibody fragment can be any single-chain antibody fragment. In other words, the antibody fragment can include a plurality of peptide chains linked to each other, for example, by disulfide bonds. The antibody fragment can also be any one of multi-molecular complexes. One functional antibody fragment typically includes at least about 50 amino acids, while more antibody fragments typically include at least about 200 amino acids. Single-chain Fvs (scFvs) are recombinant antibody fragments consisting only of a variable region of light chain (V_{L}) and a variable region of heavy chain (V_{H}) covalently bound to each other in a polypeptide chain. One of V_{L} and V_{H} has an amino-terminal region. The length and composition of polypeptide chains are variable, and their length can bridge two variable domains to each other without significantly affecting the arrangement of atoms. Polypeptide chains are typically composed mainly of glycine and serine residue extensions, with some glutamic acid and lysine residues scattered to increase their solubility. The “dimer” refers to a bipolymer of single-stranded Fvs. The monomers of the dimer typically include peptide chains that are shorter than those of most single-chain Fvs, and they show a tendency to form the bipolymer.

The “Fv” fragment consists of one V_{H} and one V_{L} domain non-covalently linked to each other. The term “dsFv” here refers to an Fv including an intermolecular disulfide bond that stabilizes a V_{H}-V_{L} pair. The “F(ab′)” fragment is a fragment of an antibody that is essentially identical to the fragment obtained by digesting an immunoglobulin (usually IgG) with pepsin at pH 4.0-4.5. This fragment can also be synthesized by recombination. The “Fab′” fragment is an antibody fragment that is essentially identical to the fragment obtained by reducing the disulfide bond linking the two heavy chains on the F(ab′) fragment. The Fab′ fragment can also be synthesized by recombination. The “Fab” fragment is an antibody fragment that is essentially identical to the fragment obtained by digesting an immunoglobulin (usually IgG) with papain. The Fab fragment can also be synthesized by recombination. The heavy chain fragment on the Fab fragment is the Fd fragment.

### BRIEF DESCRIPTION OF THE DRAWINGS

FIG. 1 is a volcano plot of differential proteins between early-stage colorectal cancer and healthy controls;

FIG. 2 is a plot showing performance analysis results of candidate markers for early-stage colorectal cancer;

FIG. 3 is an analysis graph of a random forest model for early-stage colorectal cancer+advanced adenomas vs. healthy controls constructed with 12 markers;

FIG. 4 is an analysis graph of a random forest model for early-stage colorectal cancer vs. healthy controls constructed with 12 markers;

FIG. 5 is an analysis graph of a random forest model for advanced adenomas vs. healthy controls constructed with 12 markers;

FIG. 6 is a volcano plot of differential proteins between advanced adenomas and healthy controls;

FIG. 7 is a plot showing performance analysis results of candidate markers for advanced adenomas;

FIG. 8 is a plot showing the importance analysis results of 18 candidate markers;

FIG. 9 is a performance analysis graph of a random forest model for early-stage colorectal cancer+advanced adenomas vs. healthy controls constructed with 7 markers;

FIG. 10 is a performance analysis graph of a random forest model for early-stage colorectal cancer vs. healthy controls constructed with 7 markers;

FIG. 11 is a performance analysis graph of a random forest model for advanced adenomas vs. healthy controls constructed with 7 markers;

FIG. 12 is a performance analysis graph of a random forest model for early-stage colorectal cancer+advanced adenomas vs. healthy controls constructed with 7 markers in a validation group;

FIG. 13 is a performance analysis graph of a random forest model for early-stage colorectal cancer vs. healthy controls constructed with 7 markers in a validation group;

FIG. 14 is a performance analysis graph of a random forest model for advanced adenomas vs. healthy controls constructed with 7 markers in a validation group;

FIG. 15 is a graph showing the performance evaluation (accuracy and consistency) results of an optimal model constructed by different marker combinations;

FIG. 16 is a plot showing the performance evaluation results of an optimal combined joint diagnostic model constructed in Example 2 in a testing group;

FIG. 17 is a diagram showing the performance evaluation results of an optimal combined joint diagnostic model constructed in Example 2 in a validation group;

FIG. 18 is a diagram showing performance evaluation (accuracy and consistency) results of an optimal model constructed based on 6 different algorithms in Example 3.

### DETAILED DESCRIPTION OF THE INVENTION

The present invention will be described in further detail below with reference to the accompanying drawings and examples. It should be pointed out that the examples described below are intended to facilitate the understanding of the present invention and do not limit it in any way. The reagents used in this example are all known products and are obtained by purchasing commercially available products.

### Example 1 Construction of Biomarkers and Models for Screening Early Carcinogenesis of Colorectum and Advanced Adenomas Using Proteomics

This example, by utilizing the method of proteomics, collected plasma samples of patients in different stages of the tumor progression of colorectal cancer (inflammatory diseases-, benign polyps, advanced adenomas, and colorectal cancer) and a healthy control population, analyzed different samples through high performance liquid chromatography-tandem mass spectrometry technology (HPLC-MS/MS), first screened proteins with significant differences between early-stage colorectal cancer and healthy controls based on orthogonal partial least squares discriminant analysis and significance analysis methods, and finally obtained 12 differential proteins with obvious associations with early-stage colorectal cancer through screening. However, in the random forest model constructed by the 12 proteins, the diagnostic efficiency for early carcinogenesis of colorectum (early-stage colorectal cancer and advanced adenomas) was low, and the diagnostic efficiency for advanced adenomas was reduced significantly. Therefore, in order to improve the diagnostic efficiency of early carcinogenesis of colorectum and advanced adenomas, differential proteins were screened for the advanced adenoma and healthy groups again, the top 10 differential proteins by importance ranking were finally obtained through screening, and 7 protein markers were obtained through screening by further using the Boruta algorithm to construct a model, which had a good risk prediction ability in the groups of early-stage colorectal cancer, advanced adenomas, and early-stage colorectal cancer+advanced adenomas (early carcinogenesis of colorectum). Moreover, according to the gradient boosting algorithm, a multi-marker joint detection model was further constructed, and the diagnostic efficiency of different models was evaluated by ROC analysis, and finally it was found that the diagnostic efficiency of the model constructed by the 7 biomarkers of the present invention was the highest, and the model can be used for efficient differential diagnosis of early-stage colorectal cancer, advanced adenomas, benign polyps, inflammatory diseases, healthy status, and other cancers.

The specific steps were as follows.

### (1). Sample Collection

Our research team collected 150 cases of early-stage colorectal cancer, 50 cases of advanced adenomas, 50 cases of inflammatory bowel diseases, 50 cases of benign polyps, and 50 healthy controls from January 2018 to December 2020. All enrolled patients signed informed consent forms. Patients with early-stage colorectal cancer, advanced adenomas and benign polyps were all diagnosed by colonoscopy and histopathology, patients with inflammatory bowel diseases (IBDs) were diagnosed by colonoscopy and laboratory examinations combined with clinical diagnosis, and healthy controls were normal people after routine physical examinations. Inclusion criteria for patients with early-stage colorectal cancer and patients with advanced adenomas:
(a) patients without a history of other malignant tumors;
(b) patients who had not received radiotherapy, chemotherapy or anti-tumor treatment; and
(c) patients without concomitant malignant tumors or autoimmune diseases.

The healthy individuals in the control group were selected from the physical examination center; and colonoscopy screening showed no intestinal lesions, tumor markers and biochemical indicators in laboratory examinations showed no abnormalities, and there was no history of malignant tumors. After obtaining informed consent, all collected plasma samples were stored in a plasma bank at −80° C.

### (2). Sample Treatment and Enzymatic Hydrolysis

Firstly, the plasma sample was centrifuged on a centrifuge for 15 minutes (15000×g), and the supernatant was taken and filtered, and then immunoaffinity chromatography was performed to extract 14 high-abundance proteins. Then a concentration tube with a cut-off molecular weight of 3 kDa was used for concentration on a centrifuge (4000×g, 1 hour). The concentrate was recovered and subjected to solution replacement (Buffer Exchange) on a centrifuge (1000×g, 2 minutes) using a desalting column with a cut-off molecular weight of 7 kDa, and the replacement solution was AEX-A (20 mM Tris, 4 M Urea, 3% isopropanol, pH 8.0). The protein concentration in the sample was determined using the BCA method (protein concentration detection method) with AEX-A as blank. According to the sample grouping in Table 1, TCEP (Thermo Scientific, CAT #77720) was added to the sample and incubated at 37° C. for 30 minutes for protein reduction. Then the corresponding 6-plex TMT reagent (Thermo Scientific, CAT #90309) was added, and the TMT labeling reaction was performed by incubation at room temperature for 1 hour in the dark. Afterwards, the sample was subjected to buffer displacement using a Zeba column (Thermo Scientific, CAT #89890), and the replacement solution was AEX-A. After mixing the sample labeled with 6-plex TMT, 2 mL of AEX-A was added to the mixed sample, resulting in a final volume of 5.5 mL. The sample was filtered using a 0.22 m filter and the 6-plex TMT-labeled sample was isolated using a 2D-HPLC system. The collected components were lyophilized and finally added with a Trypsin-Lysin C mixed enzyme (Thermo Scientific, CAT #A41007), and incubated at 37° C. for 5 hours for enzymatic hydrolysis of the sample, and 5 μL of 10% TFA (trifluoroacetic acid) was added to terminate the enzymatic hydrolysis reaction. A total of 60 2D-HPLC components after enzymatic hydrolysis were used for nano-LC-MS/MS analysis.

TABLE 1
Sample grouping for proteomics research (40
batches, taking batch 1 as an example)

| Sample number | Sample grouping | Experimental batch | TMT-6plex |
|---|---|---|---|
| Control | Control | Batch1 | 126 |
| Case 1 | Case | Batch1 | 127 |
| Case 2 | Case | Batch1 | 128 |
| Case 3 | Case | Batch1 | 129 |
| Case 4 | Case | Batch1 | 130 |
| Case 5 | Case | Batch1 | 131 |

### (3). LC-MS/MS Data Acquisition and Database Search Analysis

The LC-MS/MS system was a combination of Easy-nLC 1200 (Thermo Scientific) and Q Exactive HFX (Thermo Scientific), and mobile phase A was an aqueous solution containing 0.1% formic acid and 2% acetonitrile; and mobile phase B was an aqueous solution containing 0.1% formic acid and 80% acetonitrile. The length of the self-made analytical column was 20 cm, and the filling material was ReproSil-Pur C 18, 1.9 μm particles from Dr. Maisch GmbH. 1 μg of peptide fragments were dissolved in the mobile phase A and separated using an EASY-nLC 1200 ultra-high performance liquid chromatography system. Liquid phase gradient setting: 0-26 min, 7-22% B; 26-34 min, 22-32% B; 34-37 min, 32-80% B; and 37-40 min, 80% B, with the liquid phase flow rate maintained at 450 nL/min.

The peptide fragments separated by the high performance liquid chromatography system were injected into a NanoFlex ion source for nebulization and then subjected to Q Exactive HF-X for mass spectrometry analysis. The ion source voltage was set to 2.1 kV, the primary mass spectrometry scanning range was set to 400-1200, and the resolution was 60,000 (MS Resolution); and the starting point of the secondary mass spectrometry scanning range was 100 m/z, and the resolution was set to 15,000 (MS2 Resolution). The data-dependent scanning (DDA) mode was set to allow the TOP 20 parent ions to enter the HCD collision cell sequentially, and then undergo secondary mass spectrometry analysis after fragmentation sequentially. The automatic gain control (AGC) was set to 5E4, the signal threshold value was set to 1E4, and the maximum injection time was set to 22 ms. In order to avoid repeated scanning of high-abundance peptide fragments, the dynamic exclusion time for tandem mass spectrometry analysis was set to 30 seconds.

The mass spectrometry data obtained by LC-MS/MS were retrieved using Maxquant (v1.6.15.0). The data type was TMT proteomics data based on secondary reporter ion quantification. The secondary spectrum used for quantification required that the proportion of parent ions in the primary spectrum was greater than 75%. Database source was Homo_sapiens_9606_proteome of the Uniprot database (release: 2021 Oct. 14, sequence: 20614), and a common pollution database was added to the database, and polluted proteins were deleted during data analysis; the enzyme digestion mode was set to Trypsin/P; the number of missed digestion sites was set to 2; and the mass error tolerance of the parent ions was set to 20 ppm and 5 ppm for the first search and the main search, respectively, and the mass error tolerance of the secondary fragment ions was 20 ppm. The fixed modification was cysteine alkylation, and the variable modification was oxidation of methionine and acetylation of the N-terminus of the protein. The FDR of protein identification and PSM identification was set to 1%.

(4). Screening of Differential Proteins with the Highest Diagnostic Efficiency in Early Carcinogenesis of Colorectum

### 1. Screening of Differential Protein Markers in Early-Stage Colorectal Cancer

A combination of univariate analysis and multivariate statistical analysis was used to screen the differential proteins between early-stage colorectal cancer and healthy groups, where the univariate analysis mainly included the significance analysis (p value or FDR value) and fold change of characteristic ions in different groups, and the multivariate statistical analysis mainly included principal component analysis (PCA), partial least squares discriminant analysis (PLS-DA) and orthogonal partial least squares discriminant analysis (OPLS-DA). Unsupervised principal component analysis can analyze the separation trend of proteins among groups; and supervised orthogonal partial least squares discriminant analysis can analyze the difference degree of proteins between groups.

A total of 3051 proteins were identified and 1631 proteins were quantified, including some newly found markers related to early-stage colorectal cancer. For the 1631 protein substances found, the protein substances with significant differences in contents were obtained through analysis. All statistical analyses were completed using R, and the specific R-related information is shown in Table 2.

TABLE 2
R used in the present invention and related information thereof

| Name | Version |
|---|---|
| R | 3.4.1 |
| Rstudio | 1.4.1717 |
| MixOmics | 6.10.9 |
| Ropls | 1.18.1 |

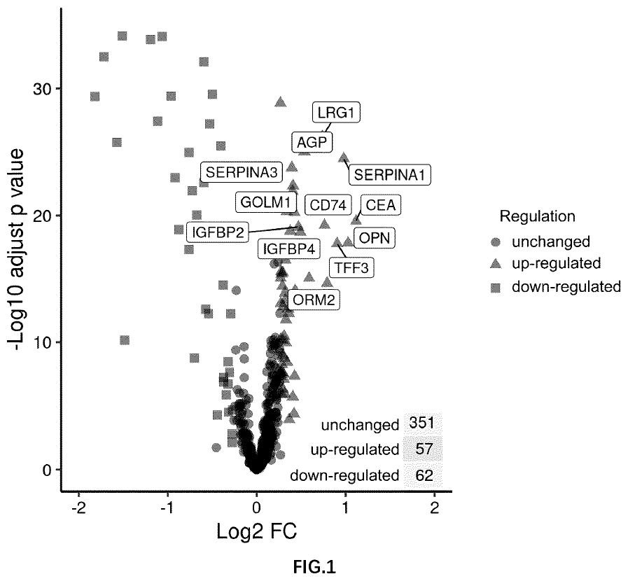

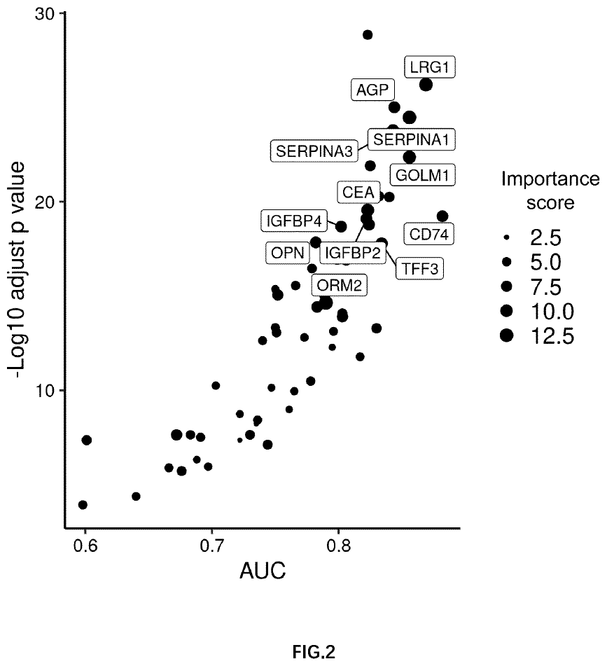

The variable importance for the projection (VIP) was calculated to measure the influence intensity and explanatory power of the expression pattern of each protein on the classification and discrimination of each group of samples, and the Wilcoxon rank sum test was further performed to obtain the corrected p value (FDR). The volcano plot results of the differential proteins between early-stage colorectal cancer and healthy controls are shown in FIG. 1: in early-stage colorectal cancer vs. healthy controls, 57 proteins were significantly upregulated and 62 proteins were significantly downregulated in the serum of patients with early-stage colorectal cancer. The performance analysis results of candidate markers for early-stage colorectal cancer are shown in FIG. 2. The abscissa was the AUC obtained by ROC analysis, the ordinate was the VIP value obtained by OPLS-DA analysis, and the size of the point represented the P value calculated by the Wilcoxon test.

The differential proteins were ranked in importance by T-test difference analysis and OPLS-DA analysis. According to the importance ranking of markers, the differential proteins ranked in the top 12 for the early-stage colorectal cancer and healthy groups were listed in this example, and the information on the 12 differential proteins is shown in Table 3. At the same time, the single diagnostic performance ROC curves of the 12 differential proteins were established respectively, and the experimental results were assessed by the area under the curve (AUC). An AUC of 0.5 indicated that a single protein had no diagnostic value; an AUC greater than 0.5 indicated that a single protein had diagnostic value; the greater the AUC, the higher the diagnostic value of a single protein; similarly, for the possible range of AUC values-95% confidence interval, the closer it was to 1, the higher and more credible the diagnostic value of the protein; and at the same time, the closer the sensitivity and specificity of the ROC were to 100%, the higher the diagnostic efficiency of this method. The cut-off value represented a specific threshold value used to distinguish positive and negative results in diagnostic tests, and when the cut-off value was too high, it may lead to an increase in false negatives and miss individuals who were truly diseased; and when the cutoff value was too low, it may lead to an increase in false positives and misidentify healthy individuals as diseased. Therefore, an appropriate cut-off value can more accurately distinguish patients from healthy individuals, thereby improving the accuracy of diagnosis. The median importance (medianImp) reflected the intermediate level of the relative importance of the differential proteins for distinguishing different groups or states in the screening of differential markers. The higher the median importance, the greater the contribution of the protein to distinguishing as a whole:

TABLE 3
12 most important differential proteins in early-stage colorectal cancer vs. healthy control groups

| | | Early-stage colorectal cancer vs. healthy control | | | | | | | |
|---|---|---|---|---|---|---|---|---|---|
| | | | | | 95% | | | Cut- | MedianImp |
| Ranking of | Differential | | | | confidence | | | off | (median |
| importance | protein name | LogFC | adj. P. Val | AUC | interval | Sensitivity | Specificity | value | importance) |
| 1 | CD74 | 0.762 | 5.89e−20 | 0.882 | 0.830- | 0.99 | 0.714 | 0.407 | 8.38 |
| | (leukocyte | | | | 0.935 | | | | |
| | differentiation | | | | | | | | |
| | antigen 74) | | | | | | | | |
| 2 | LRG1 | 0.738 | 5.99e−27 | 0.869 | 0.821- | 0.992 | 0.708 | 0.381 | 12.46 |
| | (leucine-rich | | | | 0.917 | | | | |
| | α2 | | | | | | | | |
| | glycoprotein | | | | | | | | |
| | 1) | | | | | | | | |
| 3 | GOLM1 | 0.408 | 4.36e−23 | 0.856 | 0.809- | 0.869 | 0.762 | 0.157 | 11.32 |
| | (Golgi | | | | 0.904 | | | | |
| | membrane | | | | | | | | |
| | protein 1) | | | | | | | | |
| 4 | SERPINA1 | 0.978 | 3.3e−25 | 0.856 | 0.803- | 0.984 | 0.72 | 0.471 | 12.58 |
| | (serine | | | | 0.909 | | | | |
| | protease | | | | | | | | |
| | inhibitor A1) | | | | | | | | |
| 5 | AGP (acid | 0.535 | 9.44e−26 | 0.844 | 0.795- | 0.885 | 0.723 | 0.282 | 8.76 |
| | glycoprotein) | | | | 0.894 | | | | |
| 6 | SERPINA3 | 0.394 | 1.69e−24 | 0.843 | 0.792- | 0.908 | 0.715 | 0.252 | 10.20 |
| | (serine | | | | 0.893 | | | | |
| | protease | | | | | | | | |
| | inhibitor A3) | | | | | | | | |
| 7 | Trifoil factor 3 | 0.904 | 1.65e−18 | 0.834 | 0.781- | 0.923 | 0.685 | 0.263 | 9.73 |
| | (TFF3) | | | | 0.886 | | | | |
| 8 | CEA | 1.116 | 2.83e−20 | 0823 | 0.759- | 0.957 | 0.739 | 0.356 | 10.64 |
| | (carcinoembryonic | | | | 0.887 | | | | |
| | antigen) | | | | | | | | |
| 9 | IGFBP2 | 0.469 | 8.01e−20 | 0.822 | 0.768- | 1 | 0.569 | 0.416 | 8.72 |
| | (insulin-like | | | | 0.875 | | | | |
| | growth factor | | | | | | | | |
| | binding | | | | | | | | |
| | protein 2) | | | | | | | | |
| 10 | IGFBP4 | 0.491 | 2.08e−19 | 0.802 | 0.744- | 0.928 | 0.664 | 0.28 | 8.61 |
| | (insulin-like | | | | 0.861 | | | | |
| | growth factor | | | | | | | | |
| | binding | | | | | | | | |
| | protein 4) | | | | | | | | |
| 11 | ORM2 | 0.791 | 2.26e−15 | 0.79 | 0.727- | 0.977 | 0.685 | 0.317 | 13.40 |
| | (orosomucoid | | | | 0.854 | | | | |
| | 2) | | | | | | | | |
| 12 | OPN | 1.027 | 1.44e−18 | 0.782 | 0.722- | 0.969 | 0.615 | 0.421 | 8.60 |
| | (osteopontin) | | | | 0.841 | | | | |

The association between the concentration changes of the 12 biomarkers and whether individuals suffered from early-stage colorectal cancer can be distinguished by the AUC value, 95% confidence interval, sensitivity, specificity, etc. in Table 3, among which the AUC value was the most intuitive and obvious one. The higher the AUC value, the more accurately the biomarker can distinguish the early-stage colorectal cancer population from the non-colorectal cancer population.

It can be seen from Table 3 that the concentration changes of the 12 biomarkers were significantly associated with whether individuals suffered from early-stage colorectal cancer. Using any one of the 12 biomarkers alone, its concentration change can be used to distinguish patients with early-stage colorectal cancer from healthy controls.

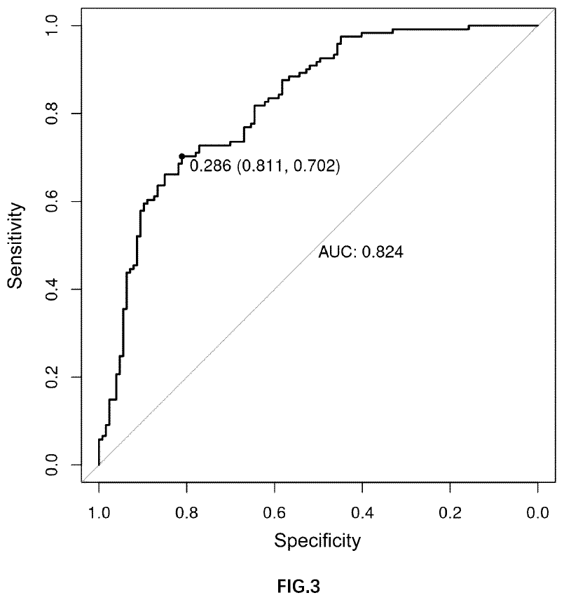

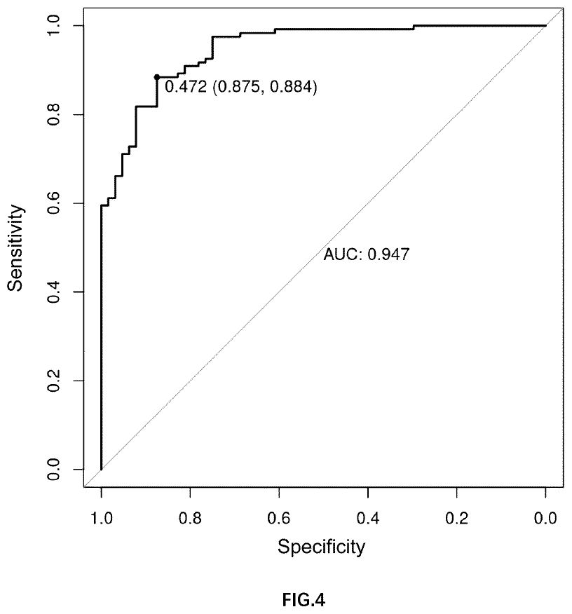

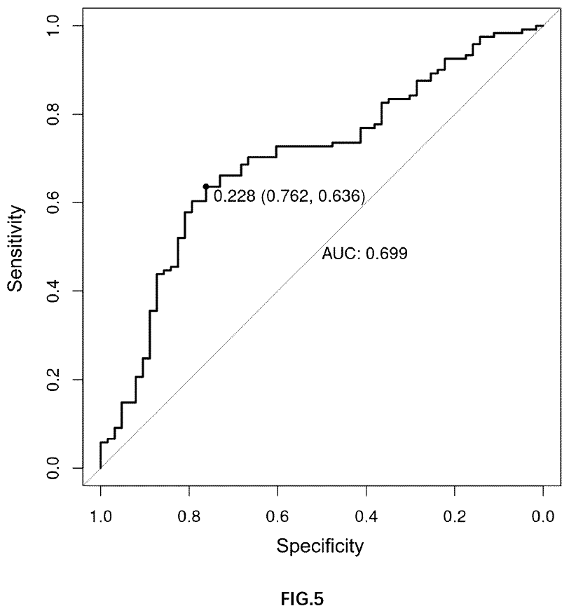

At the same time, the 12 candidate protein markers for colorectal cancer were further verified by adopting ELISA (enzyme-linked immunosorbent assay), specifically including the blood samples of 64 cases of early-stage colorectal cancer, 63 cases of advanced adenomas and 121 healthy controls. The random forest algorithm was adopted to construct a model composed of the 12 markers. The final performance of the model is shown in FIGS. 3-5, where FIG. 3 is an analysis graph of the random forest model for early-stage colorectal cancer+advanced adenomas vs. healthy controls constructed with the 12 markers, FIG. 4 is an analysis graph of the random forest model for early-stage colorectal cancer vs. healthy controls constructed with the 12 markers, and FIG. 5 is an analysis graph of the random forest model for advanced adenomas vs. healthy controls constructed with the 12 markers. It can be seen from FIGS. 3 to 5 that the model constructed by using the 12 protein markers screened in the early-stage colorectal cancer and healthy control groups had a better risk prediction ability in the early-stage colorectal cancer group (AUC=0.947), and a decreased risk prediction ability in the early-stage colorectal cancer+advanced adenoma (early carcinogenesis of colorectum) group (AUC=0.824). However, in advanced adenomas, the predictive performance was significantly reduced (AUC=0.699), and the AUC was lower than 0.8, so it was impossible to effectively diagnose advanced adenomas, and when the model was used for early carcinogenesis of colorectum (including early-stage colorectal cancer+advanced adenomas), the diagnostic value was also low.

### 2. Re-Screening of Differential Protein Markers in Advanced Adenomas

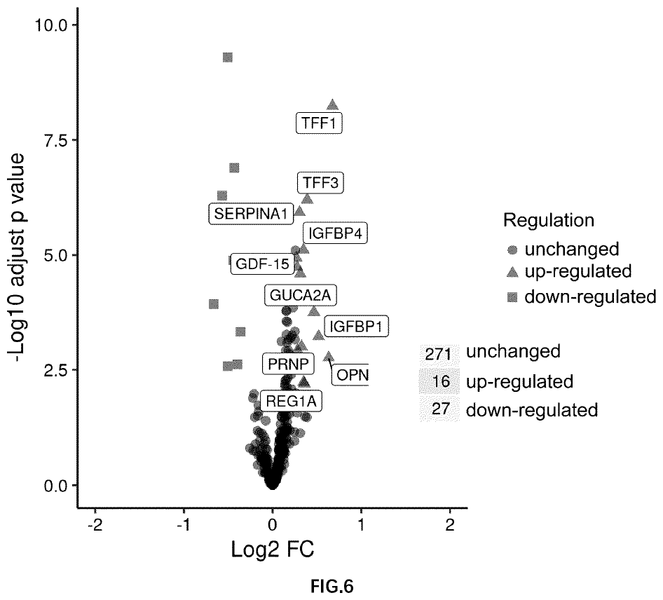

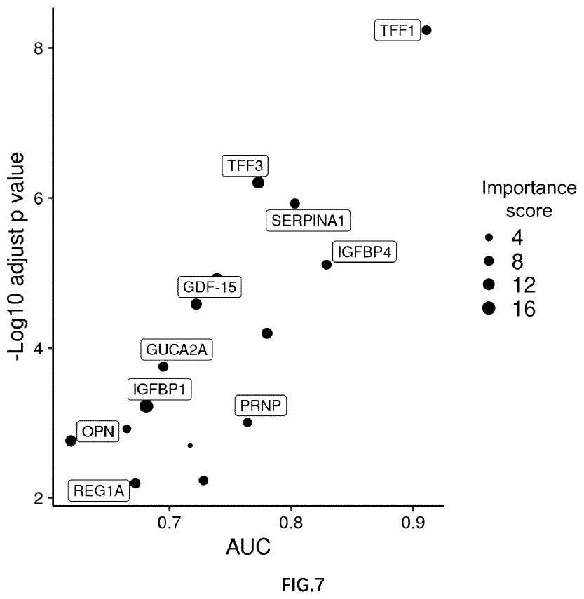

Therefore, in order to improve the diagnostic efficiency for patients with early carcinogenesis of colorectum (early-stage colorectal cancer+advanced adenomas) and patients with advanced adenomas, in this example, the differential protein screening was performed again for advanced adenoma and healthy groups. The mass spectrometry platform-based TMT labeling quantification technology strategy was used for the discovery study of early protein markers of colorectal cancer. The study cohort consisted of blood samples from 50 healthy controls and 50 patients with advanced adenomas. Through T-test difference analysis and OPLS-DA analysis, candidate markers were screened out, and the specific results are shown in FIG. 6 to FIG. 7.

As can be seen from FIG. 6, in the advanced adenoma vs. healthy groups, 16 proteins were significantly upregulated and 27 proteins were significantly downregulated in the serum of patients with advanced adenomas. The results of ROC and OPLS-DA analysis are shown in FIG. 7. The abscissa was the AUC obtained by ROC analysis, the ordinate was the VIP value obtained by OPLS-DA analysis, and the size of the point represented the P value obtained by the Wilcoxon test. At the same time, the differential proteins were ranked in importance. According to the importance ranking of markers, in this example, the differential proteins ranked in the top 10 for the advanced adenoma and healthy groups were listed respectively, and the information on the 10 differential proteins is shown in Table 4.

TABLE 4
10 most important differential proteins in advanced adenoma vs. healthy control groups

| | | Early-stage colorectal cancer vs. healthy control | | | | | | | |
|---|---|---|---|---|---|---|---|---|---|
| | Differential | | | | 95% | | | Cut- | MedianImp |
| Ranking of | protein | | | | confidence | | | off | (median |
| importance | name | LogFC | adj. P. Val | AUC | interval | Sensitivity | Specificity | value | importance) |
| 1 | TFF1 | 0.674 | 5.75e−9 | 0.911 | 0.840- | 0.871 | 0.871 | 0.27 | 7.03 |
| | (trefoil | | | | 0.981 | | | | |
| | factor 1) | | | | | | | | |
| 2 | IGFBP4 | 0.347 | 0.00000771 | 0.829 | 0.734- | 0.861 | 0.694 | 0.121 | 7.66 |
| | (insulin- | | | | 0.923 | | | | |
| | like growth | | | | | | | | |
| | factor | | | | | | | | |
| | binding | | | | | | | | |
| | protein 4) | | | | | | | | |
| 3 | SERPINA1 | 0.303 | 0.00000118 | 0.803 | 0.712- | 0.86 | 0.651 | 0.173 | 7.74 |
| | (serine | | | | 0.894 | | | | |
| | protease | | | | | | | | |
| | inhibitor | | | | | | | | |
| | A1) | | | | | | | | |
| 4 | TFF3 | 0.389 | 6.24e−7 | 0.773 | 0.674- | 0.957 | 0.587 | 0.307 | 11.97 |
| | (trefoil | | | | 0.872 | | | | |
| | factor 3) | | | | | | | | |
| 5 | PRNP | 0.326 | 0.000988 | 0.764 | 0.639- | 1 | 0.545 | 0.341 | 6.08 |
| | (prion | | | | 0.889 | | | | |
| | protein) | | | | | | | | |
| 6 | GDF-15 | 0.312 | 0.000026 | 0.722 | 0.610- | 0.935 | 0.587 | 0.3 | 10.04 |
| | (growth | | | | 0.835 | | | | |
| | differentiation | | | | | | | | |
| | factor-15) | | | | | | | | |
| 7 | GUCA2A | 0.465 | 0.000176 | 0.695 | 0.565- | 0974 | 0.526 | 0.321 | 7.55 |
| | (guanylate | | | | 0.826 | | | | |
| | cyclase | | | | | | | | |
| | activator | | | | | | | | |
| | 2A) | | | | | | | | |
| 8 | IGFBP1 | 0.518 | 0.000594 | 0.681 | 0.559- | 0.957 | 0.587 | 0.228 | 17.05 |
| | (insulin- | | | | 0.804 | | | | |
| | like growth | | | | | | | | |
| | factor | | | | | | | | |
| | binding | | | | | | | | |
| | protein 1) | | | | | | | | |
| 9 | REG1A | 0.359 | 0.00637 | 0.672 | 0.558- | 0.978 | 0.413 | 0.298 | 8.09 |
| | (regenerating | | | | 0.787 | | | | |
| | family | | | | | | | | |
| | member | | | | | | | | |
| | protein 1α) | | | | | | | | |
| 10 | OPN | 0.631 | 0.00173 | 0.619 | 0.497- | 0.978 | 0.435 | 0.401 | 10.00 |
| | (osteopontin) | | | | 0.740 | | | | |

The association between the concentration changes of the 10 differential proteins and whether individuals suffered from advanced adenomas can be distinguished by the AUC value, 95% confidence interval, sensitivity, specificity, etc. in Table 4, among which the AUC value was the most intuitive and obvious one. The higher the AUC value, the more accurately the differential protein can distinguish between the advanced adenoma population and the healthy population.

It can be seen from Table 4 that the concentration changes of the 10 differential proteins were significantly associated with whether individuals suffered from advanced adenomas. Using any one of the 10 differential proteins alone, its concentration change can be used to distinguish patients with advanced adenomas from healthy controls.

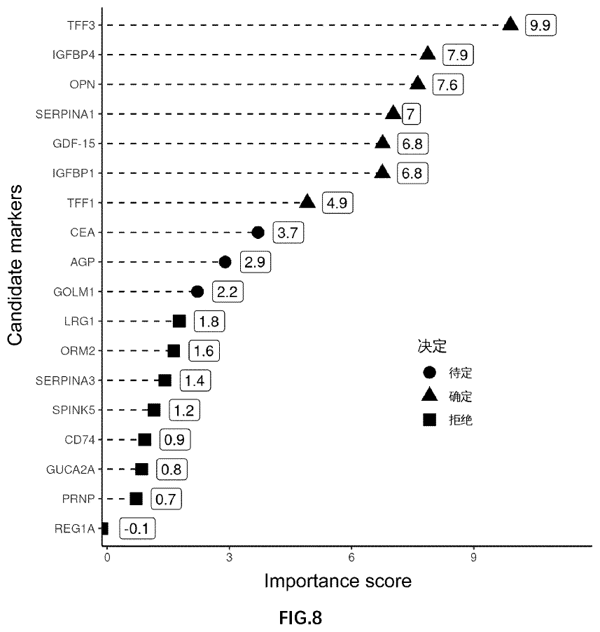

At the same time, in this example, a total of 18 candidate protein markers for colorectal cancer, the 12 candidate protein markers screened for early-stage colorectal cancer and the 10 candidate protein markers screened for advanced adenomas, were subjected to cohort validation by adopting ELISA. The cohort included the blood samples of 327 cases with early-stage colorectal cancer, 322 cases with advanced adenomas and 605 healthy controls. The importance of the 18 candidate markers was evaluated by the Boruta algorithm, and 7 markers with significant contributions to the model were finally screened and used to construct a final model. The specific results are shown in FIG. 8. As can be seen from FIG. 8, in this example, the 7 markers with significant contributions to the model obtained by the final screening were: TFF3, IGFBP4, OPN, SERPINA1, GDF-15, IGFBP1, and TFF1.

The parameters of the random forest model are specifically shown in Table 5.

TABLE 5
Random forest model parameters

| | | | n. minobsinnode |
|---|---|---|---|
| n. tress (number of | interaction. depth | Shrinkage (learning | (minimum number of |
| random forest trees) | (maximum tree depth) | rate) | leaf nodes) |
| 150 | 3 | 0.1 | 10 |

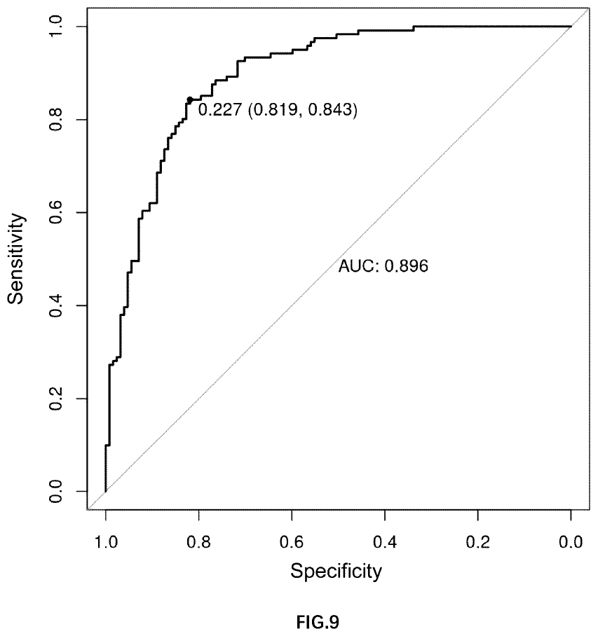

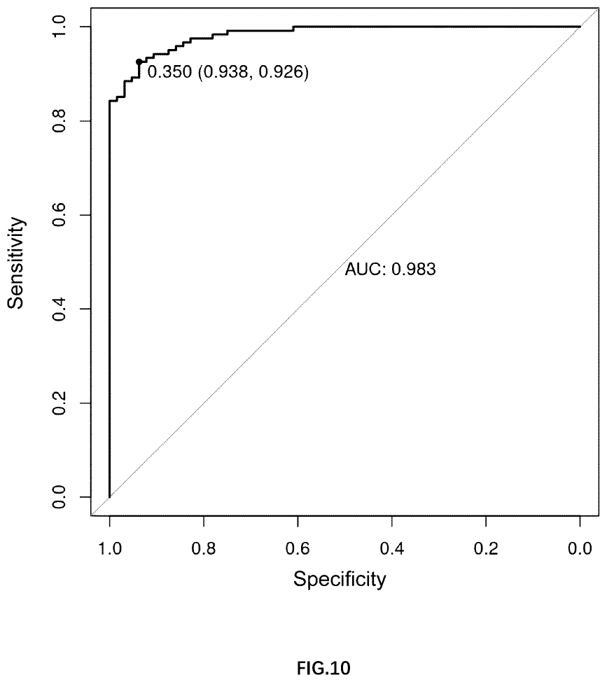

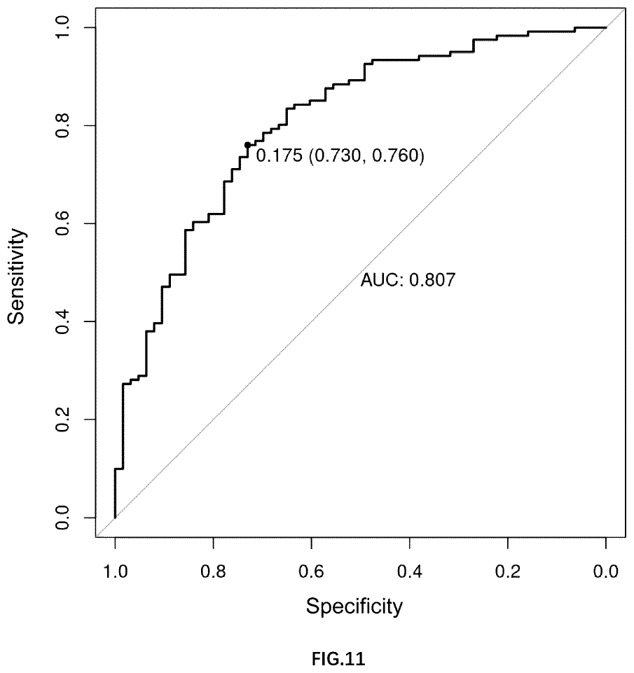

The final performance of the model composed of the 7 markers constructed by the random forest algorithm is shown in FIGS. 9 to 11, where FIG. 9 is a performance analysis graph of the random forest model for early-stage colorectal cancer+advanced adenomas vs. healthy controls constructed with the 7 markers, FIG. 10 is a performance analysis graph of the random forest model for early-stage colorectal cancer vs. healthy controls constructed with the 7 markers, and FIG. 11 is a performance analysis graph of the random forest model for advanced adenomas vs. healthy controls constructed with the 7 markers. It can be seen from FIGS. 9 to 11 that the 7 protein markers obtained by screening were used to construct the model, which had a good risk prediction ability in the early-stage colorectal cancer, advanced adenoma and early-stage colorectal cancer+advanced adenoma (early carcinogenesis of colorectum) groups, where in the early carcinogenesis of colorectum vs healthy control groups, the AUC value of the model reached 0.896; in the early-stage colorectal cancer vs healthy control groups, the model AUC value reached 0.983; and in the advanced adenoma vs healthy control groups, the AUC value of the model reached 0.807, all the AUC values reached above 0.8, and the diagnostic performance for early carcinogenesis of colorectum (including advanced adenomas and early-stage colorectal cancer) was also significantly improved.

To sum up, it can be seen that the 7 markers that contributed significantly to the model obtained by the final screening were all differential markers included in advanced adenomas, while the optimal 7 markers that contributed significantly to the model of the present invention cannot be obtained by screening only from the protein markers of early-stage colorectal cancer.

### 3. Performance Validation of a Model Constructed by 7 Protein Markers

In this example, the ELISA method was also adopted to further carry out independent cohort validation on the screened 7 candidate protein markers of colorectal cancer. The cohort included the blood samples of 86 cases of early-stage colorectal cancer, 130 cases of advanced adenomas and 173 healthy controls, and a random forest model constructed in a training cohort was used for validation.

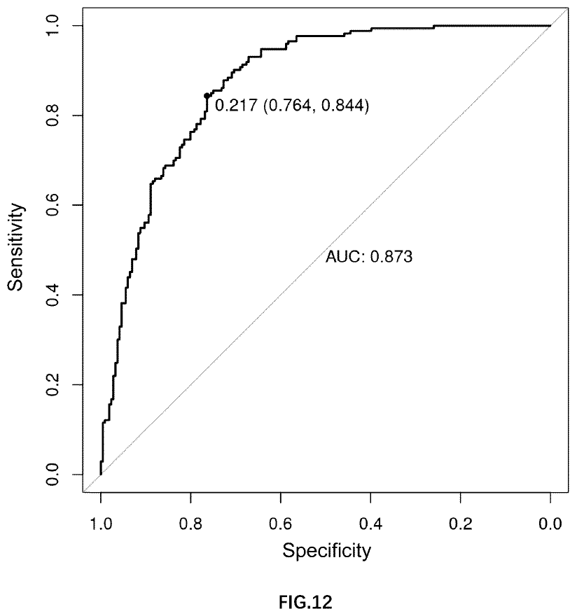

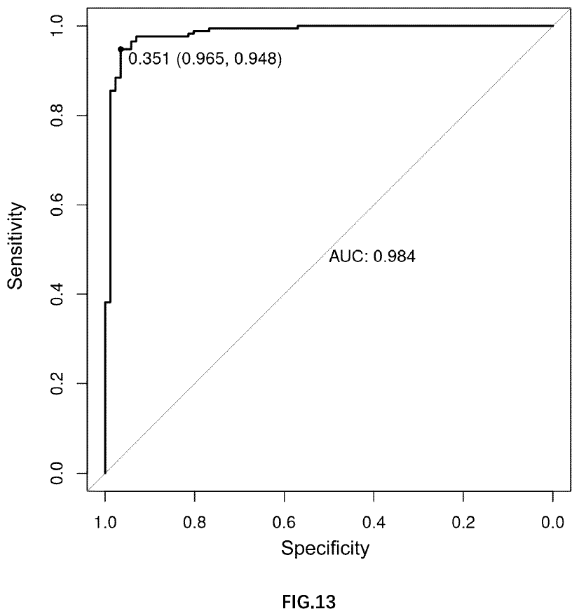

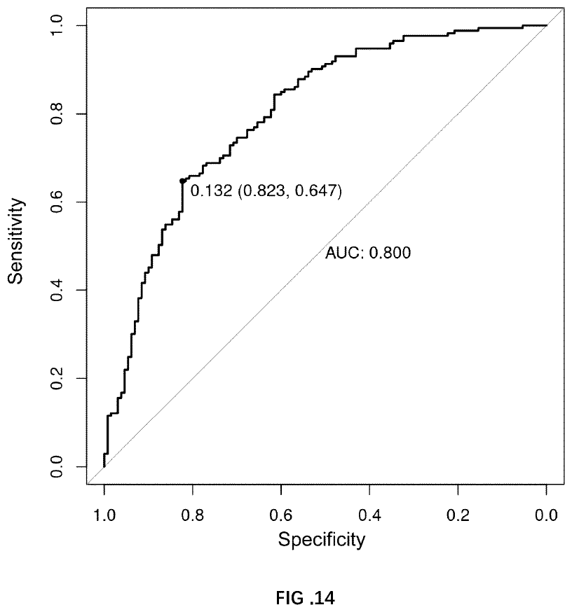

The specific results are shown in FIGS. 12 to 14, where FIG. 12 is a performance analysis graph of the random forest model for early-stage colorectal cancer+advanced adenomas vs. healthy controls constructed with the 7 markers in the validation group, FIG. 13 is a performance analysis graph of the random forest model for early-stage colorectal cancer vs. healthy controls constructed with the 7 markers in the validation group, and FIG. 14 is a performance analysis graph of the random forest model for advanced adenomas vs. healthy controls constructed with the 7 markers in the validation group. It can be seen from FIGS. 12 to 14 that the predicted results in the validation group were highly consistent with actual clinical diagnosis results, and the model had a good risk prediction ability in the early-stage colorectal cancer, advanced adenoma and early-stage colorectal cancer+advanced adenoma (early carcinogenesis of colorectum) groups, where in the early carcinogenesis of colorectum vs healthy control groups, the AUC value of the model reached 0.873; in the early-stage colorectal cancer vs healthy control groups, the model AUC value reached 0.984; and in the advanced adenoma vs healthy control groups, the AUC value of the model reached 0.800, all the AUC values reached 0.8 and above, and the diagnostic performance for early carcinogenesis of colorectum (including advanced adenomas and early-stage colorectal cancer) was also significantly improved.

Therefore, it can be seen that the diagnostic model constructed by adopting the 7 markers in the present invention had better predictive performance and accuracy, and had the optimal diagnostic efficiency.

It was confirmed that the specific information on the 7 novel biomarkers (TFF1, TFF3, IGFBP1, IGFBP4, SERPINA1, OPN, GDF-15) that met the criteria and had significant differences and high importance was as follows: trefoil factor 1 (TFF1) is a protein or an amino acid sequence with a UniProt database number of P04155; trefoil factor 3 (TFF3) is a protein or an amino acid sequence with a UniProt database number of Q07654; the insulin-like growth factor binding protein 1 (IGFBP1) is a protein or an amino acid sequence with a UniProt database number of P08833; the insulin-like growth factor binding protein 4 (IGFBP4) is a protein or an amino acid sequence with a UniProt database number of P22692; the serine protease inhibitor A1 (SERPINA1) is a protein or an amino acid sequence with a UniProt database number of P01009; the osteopontin (OPN) is a protein or an amino acid sequence with a UniProt database number of P10451; and the growth differentiation factor-15 (GDF-15) is a protein or an amino acid sequence with a UniProt database number of Q99988.

### Example 2 Construction and Validation of Senary Classification Diagnostic Models

Therefore, in this example, the combinations of the seven different biomarkers obtained by screening in Example 1 were selected to construct senary classificationsenary classification combined diagnostic models. These models were used to distinguish early carcinogenesis of colorectum, early-stage colorectal cancer, advanced adenomas, benign polyps, inflammatory bowel diseases, and healthy status, including the following processes: (1) construction and screening of the optimal diagnostic model; and (2) effect validation of the optimal diagnostic model. The specific screening processes and results were as follows (in the present invention, the binary classification model adopted a random forest construction model, and used an AUC value as an evaluation index. However, when senary classifications were adopted to construct a model, since multiple categories were involved, the AUC value was usually not applicable. Therefore, in the present invention, indexes such as accuracy, consistency, sensitivity, and specificity were adopted to measure the diagnostic efficiency of the models):

### (1). Construction and Screening of the Optimal Diagnostic Model

### 1. Data Acquisition

### Study Population:

A testing cohort with 1962 cases of colorectal cancer and a validation cohort with 390 cases of colorectal cancer were collected from September 2022 to March 2023, and all enrolled patients signed informed consent forms. Patients with early-stage colorectal cancer, advanced adenomas, benign polyps, and other cancers were all diagnosed by colonoscopy and histopathology, patients with inflammatory bowel diseases (IBDs) were diagnosed by colonoscopy and laboratory examinations combined with clinical diagnosis, and healthy controls were people with normal routine physical examinations, and negative tumor markers and fecal occult blood tests. In the testing group, early-stage colorectal cancer n=321, advanced adenomas n=321, benign polyps (BPs) n=226, inflammatory bowel diseases n=299, healthy controls n=602, other cancers n=193), and in the validation group (early-stage colorectal cancer n=64, advanced adenomas (BPs) n=64, benign polyps n=43, inflammatory bowel diseases n=60, healthy controls n=120, other cancers n=39). The data information is shown in Table 6 (in this example, the other cancers include other digestive tract cancers, such as esophageal cancer, gastric cancer, liver cancer, pancreatic cancer, bile duct cancer, etc. The senary classification detection model of the present invention can not only accurately distinguish early carcinogenesis of colorectum from early-stage colorectal cancer, advanced adenomas, benign polyps, inflammatory bowel diseases, and healthy status, but also show significant advantages in distinguishing early carcinogenesis of colorectum from other digestive tract cancers (such as esophageal cancer, gastric cancer, liver cancer, pancreatic cancer, bile duct cancer, etc.)):

TABLE 6
Modeling sample information

| Grouping | Testing group | Validation group |
|---|---|---|
| Early-stage colorectal cancer | 321 | 64 |
| Advanced adenomas | 321 | 64 |
| Benign polyps | 226 | 43 |
| Inflammatory bowel diseases | 299 | 60 |
| Healthy controls | 602 | 120 |
| Other cancers | 193 | 39 |

Inclusion criteria for patients with early-stage colorectal cancer: (a) patients without a history of other malignant tumors, (b) patients undergoing surgical treatment within one month after blood collection, and with colorectal cancer confirmed by postoperative pathology. The healthy individuals in the control group were selected from the physical examination center; and these individuals were confirmed by endoscopy to have no indication of gastric diseases and no history of malignant tumors. After obtaining informed consent, all collected serum samples were stored in a serum bank at −80° C.

In this example, the enzyme-linked immunosorbent assay was performed on the collected serum samples to obtain the respective concentrations of the seven protein markers of TFF1, TFF3, IGFBP1, IGFBP4, SERPINA1, OPN, and GDF-15.

### 2. Statistical Analysis of Experimental Data

The Shapiro-Wilk test was used to assess the normal distribution, and the differences in respective blood marker concentrations between colorectal cancer patients and healthy controls in the model and testing groups were analyzed using the non-parametric test Wilcoxon test. In the model group, a combination of multiple machine learning methods was used to construct a joint diagnostic model of the 7 colorectal cancer markers. The area under the receiver operator characteristic (ROC) curve (AUC) was estimated with a 95% confidence interval (CI) using a predicted probability value to assess the discriminative power of the multivariate diagnostic model. Using the testing group, the Youden index (YI) was calculated to determine the predicted probability cut-off value used to distinguish colorectal cancer patients from normal controls. Furthermore, the ROC curves for individual markers and different subgroups were constructed and compared. Standard descriptive statistics such as frequency, mean, median, positive predictive value (PPV), negative predictive value (NPV), and standard deviation (SD) were calculated to describe the experimental results of the study population. Statistical analyses were performed using R 3.6.1 and a p value less than 0.05 was considered statistically significant.

### 3. Construction Steps of Senary Classificationsenary Classification Combined Diagnostic Models (7MP)

### (1) Preliminary Comparison and Screening of Supervised Classification Algorithm Models to Construct the Optimal Diagnostic Model

In this example, in order to screen and obtain the optimal supervised classification algorithm for constructing the prediction model, the concentration matrix of the optimal 7 protein markers was used as an original training data set, and models under different supervised classification algorithms were constructed according to the following steps, and the performance of the different constructed models was compared to screen and obtain the optimal supervised classification algorithm. The specific process was as follows:

S101, using the concentration matrix of the seven protein markers of TFF1, TFF3, IGFBP1, IGFBP4, SERPINA1, OPN, and GDF-15 of the samples in the model group as the original training data set.

S102, setting the supervised classification algorithm used to construct the prediction model and the grid search range in the hyperparameter optimization process of the algorithm. Supervised classification algorithms included 6 algorithms: gradient boosting, Naive Bayes, support vector machine, neural network, generalized linear algorithm, and discriminant analysis. In this step, the grid search range of hyperparameter optimization of the model was set for each algorithm, as shown in Table 7 below.

TABLE 7
Parameter grid search ranges of 6 algorithms

| Algorithm | Parameter | Value |
|---|---|---|
| Discriminant analysis (mda) | subclasses | 2, 3, 4 |
| Gradient boosting (gbm) | interaction. depth | 1, 2, 3 |
| | n. trees | 50, 100, 150 |
| | shrinkage | 0.1 |
| | n. minobsinnode | 10 |
| generalized linear (glmnet) | alpha | 0.1, 0.55, 1 |
| | lambda | 0.002, 0.003, 0.005 |
| Naive Bayes (naïve_bayes) | usekernel | 1.0 |
| | laplace | 0 |
| | adjust | 1 |
| Neural network (avNNet) | size | 1, 3, 5 |
| | decay | 0, 0.1, 1e−04 |
| | bag | 0 |
| Support vector machine | sigma | 13.93717949 |
| (svmRadial) | C | 0.25, 0.5, 1 |

S103, according to the algorithm and the hyperparameter setting range set in step S102, selecting one of the algorithms and the corresponding hyperparameter combination mode as parameters for constructing the prediction model.

S104, dividing the original data set into K subsets according to the K-fold cross-validation mechanism. In order to ensure that the proportion of majority samples and minority samples in each subset was the same as that of the original data set, a stratified K-fold cross-validation mechanism needed to be used for data segmentation.

S105, according to the K training data subsets obtained by segmentation in step S104, selecting one of the subsets as a validation set Ddev.

S106, merging the training data subsets not selected in step S105 to form a training data set D.train.

S107, according to the training data set D.train obtained in step S106, constructing a prediction model based on the selected supervised classification algorithm and hyperparameters.

S108, according to the prediction model obtained in step S107, performing evaluation in the validation set D.dev to obtain an AUC value, and storing the prediction model and the corresponding AUC value in the prediction model pool Pool. In step S108, according to the prediction model obtained in step S107, the evaluation was performed on the validation set determined in the current iteration, and both the model and the evaluation result were stored in the prediction model pool for selection and use by the prediction model in the future. The evaluation mentioned in this step can be the AUC value or other reasonable indicators to evaluate the performance of the model.

S109, determining whether each subset was used as a validation set. In step S109, it was determined whether all the K subsets obtained in step S104 were used as a validation set and used for model training. If all the subsets were used as a validation set and training was completed, step S110 was performed; and if there was a subset not used as a validation set, step S105 was performed. This step ensured that each sample in the original data set was used as a validation set, improving the stability of the model and preventing the model from being overfitted to a certain subset.

S110, using the average AUC of all models in the predicted model pool Pool as the final performance evaluation value of the combined model. The model parameters and the final performance evaluation AUC value were stored in the optimal model pool Pool.best.

S111, determining whether each algorithm and all corresponding hyperparameter combination modes all constructed a prediction model. In step S111, it was determined whether the prediction model was constructed for all algorithms and corresponding hyperparameter combination modes obtained in step S102. If all the combination modes completed the construction of a model, step S112 was performed; and if there was a combination mode not completing the construction of a model, step S103 was performed.

S112, from the optimal model pool Pool.best obtained after the iteration of step S111, selecting the prediction model with the highest AUC value for each algorithm, and storing the candidate prediction model set M.set for colorectal cancer diagnosis.

S113, for the model set M.set obtained in step S112, evaluating the AUC value in the testing group D.test. The model with the largest AUC value was used as the final prediction model for colorectal cancer diagnosis.

Through the above model construction steps, the optimal models under 6 different algorithms were finally obtained. The 10-fold cross-validation method was used in the modeling process, and the performance of the model was evaluated by accuracy, consistency, sensitivity, specificity, etc.

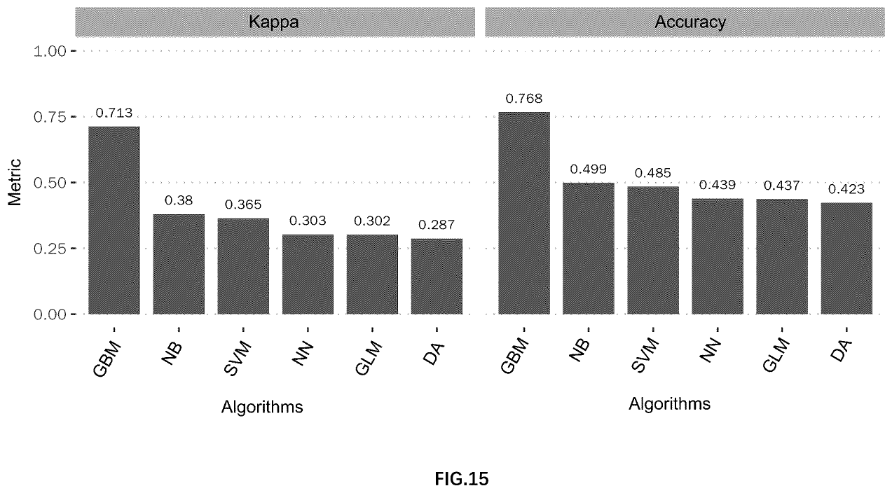

In the present invention, the testing group and the validation group adopted two completely different batches of samples, the testing group had known samples, and the inventor only screened markers from the testing group; and the samples of the validation panel were only used to validate the diagnostic efficacy of the marker combination of the present invention. The specific results are shown in Table 8 and FIG. 15: the performance evaluation scores of the gradient boosting (gbm) algorithm were all the best (for each disease type prediction, the comprehensive diagnostic accuracy was 0.768, and consistency was 0.713).

TABLE 8
Performance evaluation table of different algorithm construction
models to distinguish different disease groups

| Algorithm | Classification | Sensitivity | Specificity | Accuracy |
|---|---|---|---|---|
| Generalized | Advanced adenomas | 0.24 | 0.92 | 0.58 |
| linear | Benign polyps | 0.19 | 0.93 | 0.56 |
| | Early-stage | 0.48 | 0.90 | 0.69 |
| | colorectal cancer | | | |
| | Healthy controls | 0.65 | 0.83 | 0.74 |
| | Inflammatory bowel | 0.32 | 0.84 | 0.58 |
| | diseases | | | |
| | Other cancers | 0.51 | 0.89 | 0.70 |
| Discriminant | Advanced adenomas | 0.23 | 0.91 | 0.57 |
| analysis | Benign polyps | 0.24 | 0.88 | 0.56 |
| | Early-stage | 0.50 | 0.91 | 0.70 |
| | colorectal cancer | | | |
| | Healthy controls | 0.60 | 0.83 | 0.72 |
| | Inflammatory bowel | 0.29 | 0.85 | 0.57 |
| | diseases | | | |
| | Other cancers | 0.48 | 0.92 | 0.70 |
| Naive Bayes | Advanced adenomas | 0.39 | 0.90 | 0.65 |
| | Benign polyps | 0.44 | 0.88 | 0.66 |
| | Early-stage | 0.47 | 0.92 | 0.70 |
| | colorectal cancer | | | |
| | Healthy controls | 0.71 | 0.84 | 0.78 |
| | Inflammatory bowel | 0.23 | 0.94 | 0.59 |
| | diseases | | | |
| | Other cancers | 0.54 | 0.91 | 0.73 |
| Neural network | Advanced adenomas | 0.17 | 0.95 | 0.56 |
| | Benign polyps | 0.22 | 0.89 | 0.56 |
| | Early-stage | 0.50 | 0.89 | 0.69 |
| | colorectal cancer | | | |
| | Healthy controls | 0.70 | 0.81 | 0.75 |
| | Inflammatory bowel | 0.23 | 0.90 | 0.57 |
| | diseases | | | |
| | Other cancers | 0.56 | 0.88 | 0.72 |
| Gradient | Advanced adenomas | 0.66 | 0.95 | 0.81 |
| boosting | Benign polyps | 0.78 | 0.95 | 0.87 |
| | Early-stage | 0.76 | 0.96 | 0.86 |
| | colorectal cancer | | | |
| | Healthy controls | 0.85 | 0.95 | 0.90 |
| | Inflammatory bowel | 0.71 | 0.95 | 0.83 |
| | diseases | | | |
| | Other cancers | 0.78 | 0.96 | 0.87 |
| Support vector | Advanced adenomas | 0.30 | 0.92 | 0.61 |
| machine | Benign polyps | 0.29 | 0.89 | 0.59 |
| | Early-stage | 0.54 | 0.92 | 0.73 |
| | colorectal cancer | | | |
| | Healthy controls | 0.63 | 0.86 | 0.74 |
| | Inflammatory bowel | 0.42 | 0.87 | 0.65 |
| | diseases | | | |
| | Other cancers | 0.58 | 0.92 | 0.75 |

Based on the above analysis results, in this example, the optimal model constructed by gradient boosting (gbm) was selected as the final prediction model of senary classification joint diagnosis, and the optimal hyperparameters of the model obtained by training through a 10-fold cross-validation method were as follows: the learning rate was 0.1, the number of decision trees (number of trees) was 150, the maximum tree depth (max depth) was 3, and the minimum number of samples for the terminal node (min samples) was 10.

### (2) Validation of Combined Performance of the Optimal Joint Diagnostic Models (7MP)

In order to further analyze and study the diagnostic value of the senary classification diagnostic models (gradient boosting) constructed based on the biomarkers of different protein combinations, the performance comparison of the diagnostic models constructed based on the biomarkers of different protein combinations was performed in the testing group in this example. The combination forms of different models are specifically shown in Table 9.

TABLE 9
Combination forms of different diagnostic models

| Number of joint | |
|---|---|
| detection combinations | Optimal combination form |
| Two-item joint | TFF3 + SERPINA1 |
| detection-2MP | |
| Three-item joint | TFF1 + IGFBP1 + IGFBP4 |
| detection-3MP | |
| Four-item joint | TFF1 + IGFBP1 + IGFBP4 + SERPINA1 |
| detection-4MP | |
| Five-item joint | TFF1 + TFF3 + IGFBP4 + SERPINA1 + OPN |
| detection-5MP | |
| Six-item joint | TFF1 + TFF3 + IGFBP1 + IGFBP4 + SERPINA1 + OPN |
| detection-6MP | |
| Seven-item joint | TFF1 + TFF3 + IGFBP1 + IGFBP4 + SERPINA1 + OPN + GDF-15 |
| detection-7MP | |

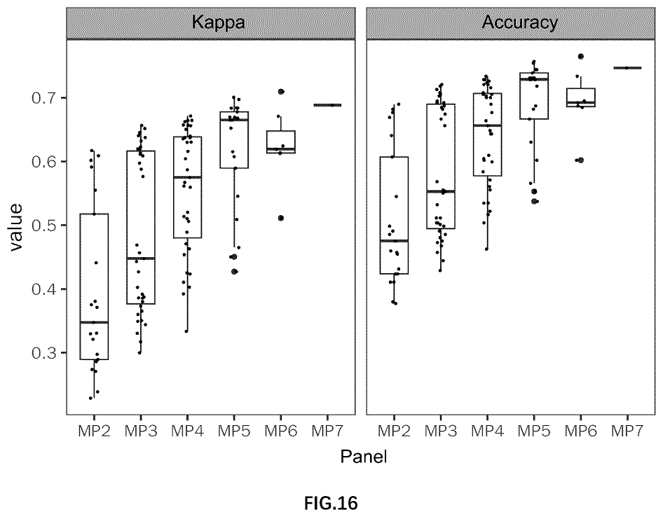

The results are specifically shown in FIG. 16 and Table 10. Table 10 shows the comparison results of performance indicators of different diagnostic models constructed by the 7 biomarkers screened in Example 1 for senary classifications. The calculation methods of the minimum value, first quartile, median value, mean value, third quartile, and maximum value of accuracy and consistency were as follows: (1) ranking the values of accuracy or consistency from small to large values; (2) minimum value: the first numerical value after ranking; (3) first quartile (Q1): multiplying the number of data by 0.25, if the result was an integer, taking the average value of the numerical values of this position and the next position; and if the result was not an integer, rounding up to get the position, and the numerical value of this position being Q1; (4) median value: if the number of data was odd, the median value being the middle numerical value; and if the number of data was even, the median value being the average of the middle two numerical values; (5) mean value: the sum of all numerical values divided by the number of data; (6) third quartile (Q3): multiplying the number of data by 0.75, and the processing method being the same as Q1; and (7) maximum value: the last numerical value after ranking.

The minimum value and the maximum value can reflect the extreme situation of the data and show the worst and best possible performance of the model; quartiles can help understand the distribution range and dispersion degree of data; below Q1 indicated a lower performance level, and above Q3 indicated a higher performance level; the median value can reflect the performance of the intermediate level; and the mean value comprehensively reflected the overall average performance. Based on the above statistical values, it can be fully understood the overall situation, distribution characteristics and stability of model performance, thus providing a strong basis for model selection and optimization.

TABLE 10
Performance comparison of diagnostic models constructed
based on biomarkers of different protein combinations

| Number of joint | Performance | Minimum | First | Median | Mean | Third | Maximum |
|---|---|---|---|---|---|---|---|
| detection combinations | indicator | value | quartile | value | value | quartile | value |
| Two-item joint | Accuracy | 0.38 | 0.42 | 0.48 | 0.51 | 0.61 | 0.69 |
| detection-2MP | Consistency | 0.23 | 0.29 | 0.35 | 0.39 | 0.52 | 0.62 |
| Three-item joint | Accuracy | 0.43 | 0.49 | 0.55 | 0.58 | 0.69 | 0.72 |
| detection-3MP | Consistency | 0.30 | 0.38 | 0.45 | 0.49 | 0.62 | 0.66 |
| Four-item joint | Accuracy | 0.46 | 0.58 | 0.66 | 0.64 | 0.71 | 0.73 |
| detection-4MP | Consistency | 0.33 | 0.48 | 0.58 | 0.56 | 0.64 | 0.67 |
| Five-item joint | Accuracy | 0.54 | 0.67 | 0.73 | 0.69 | 0.74 | 0.76 |
| detection-5MP | Consistency | 0.43 | 0.59 | 0.67 | 0.62 | 0.68 | 0.70 |
| Six-item joint | Accuracy | 0.60 | 0.69 | 0.69 | 0.69 | 0.71 | 0.76 |
| detection-6MP | Consistency | 0.51 | 0.61 | 0.62 | 0.62 | 0.65 | 0.71 |
| Seven-item joint | Accuracy | 0.78 | 0.78 | 0.78 | 0.78 | 0.78 | 0.78 |
| detection-7MP | Consistency | 0.73 | 0.73 | 0.73 | 0.73 | 0.73 | 0.73 |

From Table 10, it can be seen that for the senary classification diagnostic model, the seven-item joint detection model (7MP) composed of the optimal 7 biomarkers of the present invention had the best performance. Therefore, the senary classification gradient boosting model constructed using these seven protein biomarkers was adopted as the optimal joint diagnostic model.

### (2). Measurement and Validation of the Diagnostic Performance of the Optimal Joint Diagnostic Models (7MP)

### 1. Diagnostic Performance Measurement of Joint Diagnostic Models (7MP)

In order to more accurately determine the diagnostic performance and threshold values of the models constructed in this example on different disease classifications, a multi-classification model using the gradient boosting machine (GBM) algorithm in the model group was used for prediction analysis in the testing group, and the prediction results were calculated as the predicted probability values of 6 classifications (healthy controls, inflammatory bowel diseases, benign polyps, advanced adenomas, early-stage colorectal cancer, and other cancers), where the classification with the largest predicted probability value was the final prediction result of the system.

The meanings and calculation methods of each indicator were as follows.

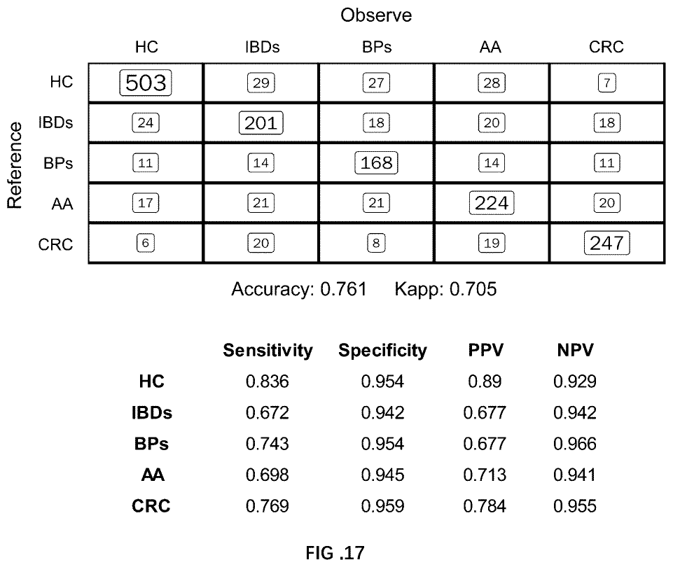

The calculation results are shown in FIG. 17: the accuracy of the model in the model group was 0.761, and the consistency was 0.705. For early-stage colorectal cancer, the diagnostic sensitivity was 76.9%, specificity was 95.9%, positive predictive value was 78.4%, and negative predictive value was 95.5%; for advanced adenomas, the diagnostic sensitivity was 69.8%, specificity was 94.5%, positive predictive value was 71.3%, and negative predictive value was 94.1%; for benign polyps, the diagnostic sensitivity was 74.3%, specificity was 95.4%, positive predictive value was 67.7%, and negative predictive value was 96.6%; for inflammatory bowel diseases, the diagnostic sensitivity was 67.2%, specificity was 94.2%, positive predictive value was 67.7%, and negative predictive value was 94.1%; for other cancers, the diagnostic sensitivity was 77.7%, specificity was 95.9%, positive predictive value was 67.3%, and negative predictive value was 97.5%; and for healthy controls, the diagnostic sensitivity was 83.6%, specificity was 95.4%, positive predictive value was 89.0%, and negative predictive value was 92.9%.

### (2). Validation of the Optimal Joint Diagnostic Models (7MP)

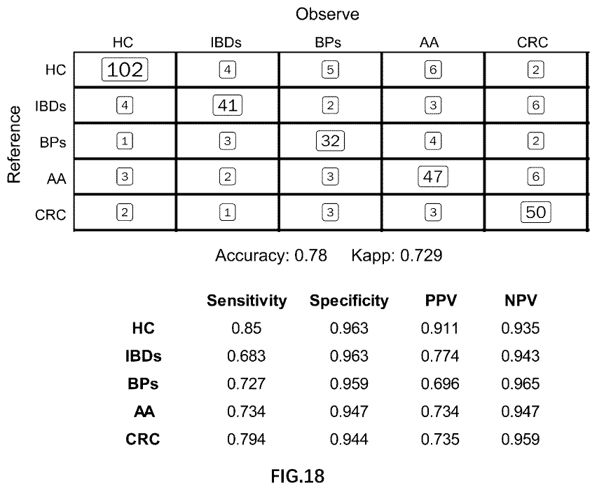

Based on the algorithm constructed by the model group, the predictive performance was validated in the validation group (Table 6). The specific results are shown in FIG. 18, with accuracy of 0.78 and consistency of 0.729. For early-stage colorectal cancer, the diagnostic sensitivity was 79.4%, specificity was 94.4%, positive predictive value was 73.5%, and negative predictive value was 95.9%; for advanced adenomas, the diagnostic sensitivity was 73.4%, specificity was 94.7%, positive predictive value was 73.4%, and negative predictive value was 94.7%; for benign polyps, the diagnostic sensitivity was 72.7%, specificity was 95.9%, positive predictive value was 69.6%, and negative predictive value was 96.5%; for inflammatory bowel diseases, the diagnostic sensitivity was 68.3%, specificity was 96.3%, positive predictive value was 77.4%, and negative predictive value was 94.3%; for other cancers, the diagnostic sensitivity was 83.3%, specificity was 96.0%, positive predictive value was 68.2%, and negative predictive value was 98.3%; and for healthy controls, the diagnostic sensitivity was 85.0%, specificity was 96.3%, positive predictive value was 91.1%, and negative predictive value was 93.5%.

To sum up, it can be seen that the joint diagnostic model including the 7 biomarkers (trefoil factor 1 (TFF1), trefoil factor 3 (TFF3), insulin-like growth factor binding protein 1 (IGFBP1), insulin-like growth factor binding protein 4 (IGFBP4), serine protease inhibitor A1 (SERPINA1), osteopontin (OPN), and growth differentiation factor-15 (GDF-15)) constructed in this example has good diagnostic value for senary classifications: early-stage colorectal cancer, advanced adenomas, benign polyps, inflammatory diseases, healthy status, and other cancers.

All patents and publications mentioned in the specification of the present invention indicate that these are disclosed techniques in the art and can be used by the present invention. All patents and publications cited herein are likewise listed in the references as if each publication is specifically and separately referenced. The present invention described herein may be implemented in the absence of any one or more elements, and one or more limitations, which are not specifically stated herein. For example, the terms “comprising”, “consisting essentially of” and “consisting of” in each of the examples herein may be replaced by the remaining two terms of one of the two. The term “one” herein only means “a”, and does not exclude the inclusion of only one, and may mean the inclusion of two or more. The terms and expressions employed herein are descriptive and are not limited thereto, and there is no intention herein to indicate that the terms and interpretations described herein exclude any equivalent features, but it can be noted that any appropriate changes or modifications can be made within the scope of the present invention and claims. It can be understood that the embodiments described in the present invention are preferred embodiments and features, and any person skilled in the art can make some modifications and changes based on the essence of the description of the present invention. These modifications and changes are also considered to be within the scope of the present invention and the scope limited by the independent claims and the dependent claims.

1. A method for diagnosing or predicting a stage of colorectal health status in an individual, comprising:
providing a biological fluid sample of the individual;
detecting the content of a marker in the biological fluid sample; and
determining the stage of the colorectal health status in the individual according to the detected content of the marker,
wherein the biomarker is selected from: any one or more of trefoil factor 1, trefoil factor 3, insulin-like growth factor binding protein 1, insulin-like growth factor binding protein 4, serine protease inhibitor A1, osteopontin, or growth differentiation factor-15.

2. The method according to claim 1, wherein the stage of the colorectal status of the individual comprises: an early-stage colorectal cancer stage, an advanced adenoma stage, a benign polyp stage, an inflammatory bowel disease stage, or a healthy stage.

3. The method according to claim 2, wherein the individual is determined to be in one of the following stages according to the detected content of the marker: the early-stage colorectal cancer stage, the advanced adenoma stage, the benign polyp stage, the inflammatory bowel disease stage a colorectal cancer stage.

4. The method according to claim 1, wherein it is determined whether the individual is in an advanced adenoma stage according to the detected content of the marker.

5. The method according to claim 1, wherein it is determined whether the individual is in an early canceration stage of colorectal cancer according to the detected content of the marker.

6. The method according to claim 5, wherein the early canceration stage of colorectal cancer comprises an advanced adenoma stage and/or an early-stage colorectal cancer stage.

7. The method according to claim 1, wherein the marker comprises the trefoil factor 1, the trefoil factor 3, the insulin-like growth factor binding protein 1, the insulin-like growth factor binding protein 4, the serine protease inhibitor A1, the osteopontin, and the growth differentiation factor-15.

8. The method according to claim 1, wherein the marker comprises a combination of the following markers: the trefoil factor 1, the trefoil factor 3, the insulin-like growth factor binding protein 1, the insulin-like growth factor binding protein 4, the serine protease inhibitor A1, the osteopontin, and the growth differentiation factor-15.

9. The method according to claim 1, wherein the biological fluid sample comprises any one of saliva, sweat, blood, urine, and spinal fluid.

10. The method according to claim 9, wherein the blood sample is whole blood, plasma or serum.

11. The method according to claim 1, wherein the detection of the biomarker in the biological fluid sample is performed using a detection reagent.

12. The method according to claim 11, wherein the detection reagent comprises an antibody or an antibody fragment that specifically binds to the marker.

13. The method according to claim 12, wherein the antibody is a monoclonal antibody.

14. The method according to claim 1, wherein the determining comprises comparing the tested content of the marker with a preset threshold value, and determining the colorectal health status of the individual according to a result of the comparison.

15. A method for predicting whether an individual suffers from an advanced adenoma, comprising:
providing a biological fluid sample of the individual;
detecting the content of a marker in the body fluid sample,
comparing the tested content of the marker with a preset threshold value; and
predicting whether the individual suffers from the advanced adenoma according to a result of the comparison,
wherein the biomarker is selected from: any one or more of trefoil factor 1, trefoil factor 3, insulin-like growth factor binding protein 1, insulin-like growth factor binding protein 4, serine protease inhibitor A1, osteopontin, growth differentiation factor-15, prion protein, guanylate cyclase activator 2A, and regenerating family member protein 1a.

16. The method according to claim 15, wherein the biomarker comprises a combination of the following markers: the trefoil factor 1, the trefoil factor 3, the insulin-like growth factor binding protein 1, the insulin-like growth factor binding protein 4, the serine protease inhibitor A1, the osteopontin, and the growth differentiation factor-15.

17. The method according to claim 15, wherein the biological fluid sample comprises any one or more of saliva, sweat, blood, urine, and spinal fluid.

18. The method according to claim 16, wherein the trefoil factor 1 is a protein or an amino acid sequence with a UniProt database number of P04155; the trefoil factor 3 is a protein or an amino acid sequence with a UniProt database number of Q07654; the insulin-like growth factor binding protein 1 is a protein or an amino acid sequence with a UniProt database number of P08833; the insulin-like growth factor binding protein 4 is a protein or an amino acid sequence with a UniProt database number of P22692; the serine protease inhibitor A1 is a protein or an amino acid sequence with a UniProt database number of P01009; the osteopontin is a protein or an amino acid sequence with a UniProt database number of P10451; and the growth differentiation factor-15 is a protein or an amino acid sequence with a UniProt database number of Q99988.

19. A kit for predicting whether an individual suffers from an advanced adenoma, comprising a detection reagent, wherein the detection reagent is used to test a content of a marker in a body fluid sample, and the marker comprises: one of trefoil factor 1, trefoil factor 3, insulin-like growth factor binding protein 1, insulin-like growth factor binding protein 4, serine protease inhibitor A1, osteopontin, and growth differentiation factor-15.

20. The kit according to claim 19, wherein the detection reagent comprises an antibody or an antibody fragment.
